(19) United States
(12) Patent Application Publication
(10) Pub. No.: US 2024/0238296 A1
(43) Pub. Date: Jul. 18, 2024

(54) METHODS OF REGULATING BCL11A EXPRESSION AND TREATMENT OF BCL11A-MEDIATED DISORDERS

(71) Applicant: Fulcrum Therapeutics, Inc., Cambridge, MA (US)

(72) Inventors: Paul Bruno, Cambridge, MA (US); Christopher Moxham, Cambridge, MA (US); Billy Stuart, Cambridge, MA (US)

(21) Appl. No.: 18/557,694

(22) PCT Filed: Apr. 28, 2022
(86) PCT No.: PCT/US2022/026795

§ 371 Date: Oct. 27, 2023

Related U.S. Application Data
(60) Provisional application No. 63/313,968, filed on Feb. 25, 2022.
(60) Provisional application No. 63/181,747, filed on Apr. 29, 2021.

(51) Int. Cl. A61K 31/519; A61K 31/437; A61K 31/472; A61P 7/00; C12Q 1/6883; G01N 33/68

(52) U.S. Cl. CPC A61K 31/519; A61K 31/437; A61K 31/472; A61P 7/00; C12Q 1/6883; G01N 33/6893; C12Q 2600/158

(57) ABSTRACT

Provided herein, in part, are methods of downregulating BCL11A expression and treatment of BCL11A mediated disorders. The methods may comprise the use of inhibitors such as EED, EHZ2, and/or PRC2 inhibitors.

### CROSS-REFERENCE

This application claims priority to U.S. Provisional Application No. 63/181,747 filed Apr. 29, 2021, and U.S. Provisional Application No. 63/313,968 filed Feb. 25, 2022, the contents of each of which are incorporated herein by reference in their entireties.

### BACKGROUND

Polycomb group (PcG) proteins are a family of chromatin modifying enzymes that play a key role in gene expression and are dysregulated in many human diseases. The PcG family includes two classes of Polycomb Repressive Complexes (PRCs), namely Polycomb Repressive Complex 1 (PRC1) and Polycomb Repressive Complex 2 (PRC2). PRC2 includes SUZ12 (suppressor of zeste 12), EED (embryonic ectoderm development) and the catalytic subunit, EZH2 (enhancer of zeste homolog 2), and represses genes by methylating histone H3 lysine 27 (H3K27me3) at and around the promoter regions of genes. This critical component of chromatin regulation is involved in modulation of gene transcription and plays crucial function in development, differentiation, and regeneration. Although EZH2 is the catalytic subunit, PRC2 minimally requires EED and SUZ12 for its methyltransferase activity. EED, SUZ12 and EZH2 have been found to be overexpressed in many cancers, which include but are not limited to hepatocellular carcinoma, breast cancer, prostate cancer, etc. Activating mutations in EZH2 have been found in FL (follicular lymphoma) and DLBCL (diffuse large B cell lymphoma) patients. EED normally mediates repression of gene activity by binding to di- and trimethylated lysine 27 of histone 3 where it allosterically activates EZH2 activity of PRC2. EED has also been reported to recruit PRC1 to H3K27me3 loci and to enhance PRC1 mediated H2A ubiquitin E3 ligase activity.

Taken together, EED is a critical regulator of PRC2 in the silencing of expression of genes and gene clusters involved in development including but not limited to fetal orthologues (i.e. gamma globin), Hox genes, X chromosome inactivation, etc. Thus, EED provides a pharmacologic target for the treatment of diseases or disorders to impact transcription of specific target genes in blood and other tissues.

A need exists for small molecules that modulate EED, EHZ2, and/or PRC2.

### SUMMARY

Provided herein, in part, are methods of downregulating BCL11A expression and treatment of BCL11A mediated disorders.

Described herein, in one embodiment, is a method of downregulating BCL11A in a cell comprising: contacting the cell sample with an EED, EHZ2, and/or PRC2 inhibitor in amount sufficient to decrease expression of BCL11A.

In another embodiment, provided herein is a method of identifying a patient having a BCL11A mediated disorder who may benefit from treatment comprising one or more inhibitors of EED, EHZ2, and/or PRC2, comprising determining an expression level of BCL11A in a sample obtained from the patient, wherein an increased expression level of BCL11A in the sample as compared to a reference expression level identifies the patient as one who may benefit from the EED, EHZ2, and/or PRC2 inhibitor treatment.

In another embodiment, provided herein is a method of treating a patient having a BCL11A mediated disorder, comprising administering to the patient a therapeutically effective amount of a EED, EHZ2, and/or PRC2 inhibitor, wherein the expression level of BCL11A in a sample obtained from the patient has been determined to be decreased after the administration as compared to a reference expression level.

In another embodiment, provided herein is a method of treatment of a hemoglobinopathy in a subject comprising administering an effective amount of a composition comprising an inhibitor of EED, EHZ2, and/or PRC2, wherein the inhibitor of EED, EHZ2, and/or PRC2 downregulates the expression of BCL11A and whereby fetal hemoglobin expression is increased in the subject relative to prior to the administration.

In another embodiment, provided herein is a genetically modified cell comprising an insertion and/or deletion in a gene loci that encodes a protein selected from the group consisting of EED, EHZ2, and PRC2, wherein the insertion and/or deletion is capable of downregulating expression of BCL11A in the cell.

### BRIEF DESCRIPTION OF THE DRAWINGS

FIG. 1 depicts changes in the mRNA expression levels of BCL11A upon treatment with Compound 1 at various concentrations.

FIG. 2 depicts fold changes in BCL11A, HBG1, and HBG2 expression in exemplary CD34+ donor cells upon treatment with 100 nM Compound 1 or 100 nM Compound 2.

FIG. 3 depicts 4-point concentration response curves of fold changes in BCL11A expression, HBG1 expression, and HBG2 expression, and percentage HbF levels, in a CD34+ donor cell (D069) upon treatment with Compound 1 at various concentrations.

FIG. 4A, FIG. 4B, and FIG. 4C depict aggregate data on the effect of HBG1 mRNA expression level (FIG. 4A), HBG2 mRNA expression level (FIG. 4B), and percentage HbF protein (FIG. 4C) from two different CD34+ donors upon treatment with multiple compounds (Compound 1, Compound 2, Compound 3, and Compound 4 as described herein) that inhibit PRC2.

FIG. 5A, FIG. 5B, and FIG. 5C depict 4-point concentration response curves of the effect of PRC2 inhibitors Compound 1, Compound 2, Compound 3, and Compound 4 on BCL11A expression (FIG. 5A), HBG1 expression (FIG. 5B), and HBG2 expression (FIG. 5C).

FIG. 6 depicts BCL11A mRNA transcript levels by qRT-PCR in wild-type CD-1 mice and in the Townes sickle cell disease (SCD) mouse model upon treatment with Compound 1.

FIG. 7 depicts BCL11A mRNA transcript levels by qRT-PCR in CD34+ cells with CRISPR knockout of EED and BCL11A with corresponding HbF levels by HPLC.

FIG. 8A depicts flow cytometry results of H3K27me3 for Compound 1 or hydroxyurea (HU) in bone marrow erythroid progenitors. FIG. 8B depicts flow cytometry results of percent F-cell in terms of percentage of vehicle for Compound 1 or HU. FIG. 8C depicts HbF HPLC from whole blood after 21 days of treatment in studies with Compound 1 or HU. FIG. 8D depicts results of Compound 1 or HU for mouse spleen weight percentage of total body weight. FIG. 8E depicts percent HbF correlations to representative hematological parameters of anemia and inflammation in studies with Compound 1 or HU.

### DETAILED DESCRIPTION

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications and this disclosure.

### Definitions

The term “alkyl” as used herein refers to a saturated straight or branched hydrocarbon. Exemplary alkyl groups include, but are not limited to, straight or branched hydrocarbons of 1-6, 1-5, 1-4, 1-3, or 1-2 carbon atoms, referred to herein as C₁-C₆ alkyl, C₁-C₅ alkyl, C₁-C₄ alkyl, C₁-C₃ alkyl, and C₁-C₂ alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂-C₆ alkenyl, and C₃-C₄ alkenyl, respectively. Exemplary alkenyl groups include, but are not limited to, vinyl, allyl, butenyl, pentenyl, etc.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁-C₆alkoxy, and C₂-C₆alkoxy, respectively. Exemplary alkoxy groups include, but are not limited to methoxy, ethoxy, isopropoxy, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂-C₆alkynyl, and C₃-C₆alkynyl, respectively. Exemplary alkynyl groups include, but are not limited to, ethynyl, propynyl, butynyl, pentynyl, hexynyl, methylpropynyl, etc.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, —halogen, —O—C₁-C₆ alkyl, C₁-C₆ alkyl, —OC₂-C₆ alkenyl, —OC₂-C₆ alkynyl, —C₂-C₆ alkenyl, —C₂-C₆ alkynyl, —OH, —OP(O)(OH)₂, —OC(O)C₁-C₆ alkyl, —C(O)C₁-C₆ alkyl, —OC(O)OC₁-C₆ alkyl, NH₂, NH(C₁-C₆ alkyl), N(C₁-C₆ alkyl)₂, —S(O)₂-C₁-C₆ alkyl, —S(O)NHC₁-C₆ alkyl, and S(O)N(C₁-C₆ alkyl)₂. The substituents can themselves be optionally substituted. Furthermore when containing two fused rings the aryl groups herein defined may have an unsaturated or partially saturated ring fused with a fully saturated ring. Exemplary ring systems of these aryl groups include indanyl, indenyl, tetrahydronaphthalenyl, and tetrahydrobenzoannulenyl.

The terms “cycloalkyl” or a “carbocyclic group” as used herein refers to a saturated or partially unsaturated hydrocarbon group of, for example, 3-6, or 4-6 carbons, referred to herein as C₃-C₆cycloalkyl or C₄-C₆cycloalkyl, respectively. Exemplary cycloalkyl groups include, but are not limited to, cyclohexyl, cyclopentyl, cyclopentenyl, cyclobutyl or cyclopropyl.

The terms “halo” or “halogen” as used herein refer to F, Cl, Br, or I.

The term “heteroaryl” as used herein refers to a monocyclic aromatic 5 or 6 membered ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl” or “heterocyclic group” are art-recognized and refer to saturated or partially unsaturated, 4-10 membered ring structures, including monocyclic, bridged or fused rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another carbocyclic, heterocyclic, aromatic, or heteroaromatic ring. One or more of the carbon atoms in the spirocycle can be substituted with a heteroatom (e.g., O, N, S, or P). A (C₅-C₁₂) spirocycloalkyl is a spirocycle containing between 5 and 12 carbon atoms. One or more of the carbon atoms can be substituted with a heteroatom.

The term “spiroheterocycloalkyl” or “spiroheterocyclyl” is understood to mean a spirocycle wherein at least one of the atoms in one of the rings is a heteroatom. In some embodiments, at least one of the atoms in one of the rings is O, N, S, or P.

The term “oxo” as used herein refers to an “═O” group.

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all enantiomeric, diastereomeric, and geometric (or conformational) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present disclosure. Unless otherwise stated, all tautomeric forms of the compounds of the present disclosure are within the scope of the present disclosure.

“Therapeutically effective amount” includes the amount of the subject compound that will elicit the biological or medical response of a tissue, system, animal or human that is being sought by the researcher, veterinarian, medical doctor or other clinician. A compound described herein, e.g., an EED, EHZ2, and/or PRC2 inhibitor, is administered in therapeutically effective amounts to treat a condition, e.g., a condition described herein. Alternatively, a therapeutically effective amount of a compound is the quantity required to achieve a desired therapeutic and/or prophylactic effect, such as an amount which results in the prevention of or a decrease in the symptoms associated with the condition.

A compound described herein, e.g., an EED, EHZ2, and/or PRC2 inhibitor, can be formulated as a pharmaceutical composition using a pharmaceutically acceptable carrier and administered by a variety of routes. In some embodiments, such compositions are for oral administration. In some embodiments, such compositions are for parenteral (by injection) administration. In some embodiments, such compositions are for transdermal administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19th ed., Mack Publishing Co., 1995). Dosages of compounds described herein in oral compositions include, but are not limited to, 6 mg.

“Compound 1” as described herein, refers to a compound represented by:

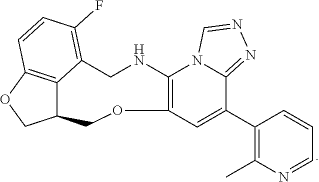 [SMILES: Cc1ncccc1-c1cc2c(n3cnnc13)NCc1c(F)ccc3c1[C@H](CO3)CO2]

“Compound 2” as described herein, refers to a compound represented by:

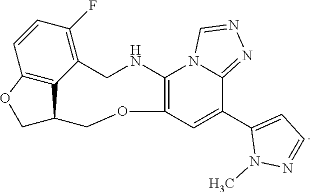 [SMILES: Cn1nccc1-c1cc2c(n3cnnc13)NCc1c(F)ccc3c1[C@H](CO3)CO2]

“Compound 3” as described herein, refers to a compound represented by:

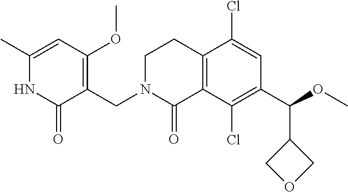

“Compound 4” as described herein, refers to a compound represented by:

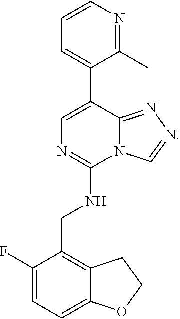 [SMILES: Cc1ncccc1-c1cnc(NCc2c(F)ccc3c2CCO3)n2cnnc12]

### Downregulation of BCL11A Expression

The present disclosure provides, in an embodiment, methods of downregulating expression of BCL11A.

A method of downregulating BCL11A described herein is, in an embodiment, a method of downregulating BCL11A in a cell comprising: contacting the cell sample with an EED, EHZ2, and/or PRC2 inhibitor in amount sufficient to decrease expression of BCL11A. In some embodiments, the cell is an erythroid cell differentiated from a CD34+ cell. In some embodiments, upon contacting the cell, HBG1 and/or HBG2 expression increases. In some embodiments, upon contacting the cell with the protein inhibitor, the cell does or does not express fetal hemoglobin. In some embodiments, the EED, EHZ2, and/or PRC2 inhibitor is selected from the group consisting of an antibody against EED, EHZ2, and/or PRC2 or an antigen-binding fragment thereof, a small molecule, and a nucleic acid. In some embodiments, the nucleic acid is a EED, EHZ2, and/or PRC2-specific RNA interference agent, a vector encoding a RNA interference agent, or an aptamer that binds EED, EZH2, and/or PRC2. In some embodiments, the expression level of BCL11A is decreased by at least 25% relative to a reference level. In some embodiments, the expression level of BCL11A is decreased by at least 50% relative to a reference level. In some embodiments, the expression level of BCL11A is decreased by at least 75% relative to a reference level. In some embodiments, the expression level of BCL11A is decreased by at least 90% relative to a reference level. In some embodiments, the expression level of BCL11A is decreased by at least 99% relative to a reference level. In some embodiments, the expression level is a protein expression level or a mRNA expression level. In some embodiments, the mRNA expression level is determined by qPCR or RNA-Seq. In some embodiments, the protein expression level is determined using a method selected from the group consisting of high-performance liquid chromatography (HPLC), immunohistochemistry (IHC), immunofluorescence, mass spectrometry, flow cytometry, and Western blot.

### Methods of Treatment

The present disclosure also provides, in another embodiment, methods of treating BCL11A mediated disorders in a patient in need thereof, comprising administering to the patient a therapeutic agent described herein, e.g., an EED, EHZ2, and/or PRC2 inhibitor. Exemplary BCL11A mediated disorders include, but are not limited to, triple negative breast cancer, non-small cell lung cancer, glioblastoma, neuroblastoma, prostate cancer, type 2 diabetes, laryngeal squamous cell carcinoma, and Williams syndrome.

Described herein, in another embodiment, is a method of identifying a patient having a BCL11A mediated disorder who may benefit from treatment comprising one or more inhibitors of EED, EHZ2, and/or PRC2, comprising determining an expression level of BCL11A in a sample obtained from the patient, wherein an increased expression level of BCL11A in the sample as compared to a reference expression level identifies the patient as one who may benefit from the EED, EHZ2, and/or PRC2 inhibitor treatment. In some embodiments, the EED, EHZ2, and/or PRC2 inhibitor is selected from the group consisting of an antibody against EED, EHZ2, and/or PRC2 or an antigen-binding fragment thereof, a small molecule, and a nucleic acid. In some embodiments, the nucleic acid is a EED, EHZ2, and/or PRC2-specific RNA interference agent, a vector encoding a RNA interference agent, or an aptamer that binds EED, EZH2, and/or PRC2. In some embodiments, the BCL11A mediated disorder is selected from the group consisting of triple negative breast cancer, non-small cell lung cancer, glioblastoma, neuroblastoma, prostate cancer, type 2 diabetes, laryngeal squamous cell carcinoma, and Williams syndrome. In some embodiments, the expression level of BCL11A is decreased by at least 25% relative to a reference level. In some embodiments, the expression level of BCL11A is decreased by at least 50% relative to a reference level. In some embodiments, the expression level of BCL11A is decreased by at least 75% relative to a reference level. In some embodiments, the expression level of BCL11A is decreased by at least 90% relative to a reference level. In some embodiments, the expression level of BCL11A is decreased by at least 99% relative to a reference level. In some embodiments, the expression level is a protein expression level or a mRNA expression level. In some embodiments, the mRNA expression level is determined by qPCR or RNA-Seq. In some embodiments, the protein expression level is determined using a method selected from the group consisting of high-performance liquid chromatography (HPLC), immunohistochemistry (IHC), immunofluorescence, mass spectrometry, flow cytometry, and Western blot.

Also described herein, in another embodiment, is a method of treating a patient having a BCL11A mediated disorder, comprising administering to the patient a therapeutically effective amount of a EED, EHZ2, and/or PRC2 inhibitor, wherein the expression level of BCL11A in a sample obtained from the patient has been determined to be decreased after the administration as compared to a reference expression level. In some embodiments, the EED, EHZ2, and/or PRC2 inhibitor is selected from the group consisting of an antibody against EED, EHZ2, and/or PRC2 or an antigen-binding fragment thereof, a small molecule, and a nucleic acid. In some embodiments, the nucleic acid is a EED, EHZ2, and/or PRC2-specific RNA interference agent, a vector encoding a RNA interference agent, or an aptamer that binds EED, EZH2, and/or PRC2. In some embodiments, the BCL11A mediated disorder is selected from the group consisting of triple negative breast cancer, non-small cell lung cancer, glioblastoma, neuroblastoma, prostate cancer, type 2 diabetes, laryngeal squamous cell carcinoma, and Williams syndrome. In some embodiments, the expression level of BCL11A is decreased by at least 25% relative to a reference level. In some embodiments, the expression level of BCL11A is decreased by at least 50% relative to a reference level. In some embodiments, the expression level of BCL11A is decreased by at least 75% relative to a reference level. In some embodiments, the expression level of BCL11A is decreased by at least 90% relative to a reference level. In some embodiments, the expression level of BCL11A is decreased by at least 99% relative to a reference level. In some embodiments, the expression level is a protein expression level or a mRNA expression level. In some embodiments, the mRNA expression level is determined by qPCR or RNA-Seq. In some embodiments, the protein expression level is determined using a method selected from the group consisting of high-performance liquid chromatography (HPLC), immunohistochemistry (IHC), immunofluorescence, mass spectrometry, flow cytometry, and Western blot.

Further described herein, in another embodiment, is a method of treatment of a hemoglobinopathy in a subject comprising administering an effective amount of a composition comprising an inhibitor of EED, EHZ2, and/or PRC2, wherein the inhibitor of EED, EHZ2, and/or PRC2 downregulates the expression of BCL11A and whereby fetal hemoglobin expression is increased in the subject relative to prior to the administration. In some embodiments, the hemoglobinopathy is selected from the group consisting of sickle cell disease (SCD), α-thalassemia, and β-thalassemia. In some embodiments, the β-thalassemia is selected from the group consisting of sickle β-thalassemia, hemoglobin C β-thalassemia, and hemoglobin E β-thalassemia. In some embodiments, the sickle β-thalassemia is selected from sickle BO thalassemia and sickle β+ thalassemia. In some embodiments, the EED, EHZ2, and/or PRC2 inhibitor is selected from the group consisting of an antibody against EED, EHZ2, and/or PRC2 or an antigen-binding fragment thereof, a small molecule, and a nucleic acid. In some embodiments, the nucleic acid is a EED, EHZ2, and/or PRC2-specific RNA interference agent, a vector encoding a RNA interference agent, or an aptamer that binds EED, EZH2, and/or PRC2. In some embodiments, the expression level of BCL11A is decreased by at least 25% relative to a reference level. In some embodiments, the expression level of BCL11A is decreased by at least 50% relative to a reference level. In some embodiments, the expression level of BCL11A is decreased by at least 75% relative to a reference level. In some embodiments, the expression level of BCL11A is decreased by at least 90% relative to a reference level. In some embodiments, the expression level of BCL11A is decreased by at least 99% relative to a reference level. In some embodiments, the expression level is a protein expression level or a mRNA expression level. In some embodiments, the mRNA expression level is determined by qPCR or RNA-Seq. In some embodiments, the protein expression level is determined using a method selected from the group consisting of high-performance liquid chromatography (HPLC), immunohistochemistry (IHC), immunofluorescence, mass spectrometry, flow cytometry, and Western blot.

### Genetically Modified Cells

The present disclosure additionally provides, in another embodiment, a genetically modified cell comprising an insertion and/or deletion in a gene loci that encodes a protein selected from the group consisting of EED, EHZ2, and PRC2, wherein the insertion and/or deletion is capable of downregulating expression of BCL11A in the cell. In some embodiments, the expression level of BCL11A is decreased by at least 25% relative to a reference level. In some embodiments, the expression level of BCL11A is decreased by at least 50% relative to a reference level. In some embodiments, the expression level of BCL11A is decreased by at least 75% relative to a reference level. In some embodiments, the expression level of BCL11A is decreased by at least 90% relative to a reference level. In some embodiments, the expression level of BCL11A is decreased by at least 99% relative to a reference level. In some embodiments, the expression level is a protein expression level or a mRNA expression level. In some embodiments, the mRNA expression level is determined by qPCR or RNA-Seq. In some embodiments, the protein expression level is determined using a method selected from the group consisting of high-performance liquid chromatography (HPLC), immunohistochemistry (IHC), immunofluorescence, mass spectrometry, flow cytometry, and Western blot.

### Inhibitors

The present disclosure also provides, in some embodiments, inhibitors useful in the methods described herein. Such inhibitors may include EED, EHZ2, and/or PRC2 inhibitors.

In one embodiment, an inhibitor described herein is represented by a compound of Formula I:

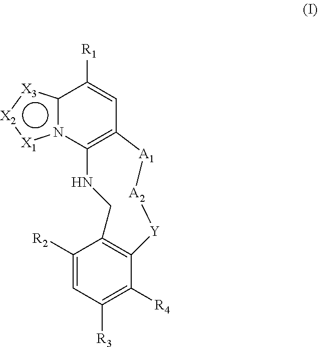

or a pharmaceutically acceptable salt, prodrug, solvate, hydrate, enantiomer, isomer, or tautomer thereof, wherein:

X₁, X₂, and X₃ are independently N or C(R₅), provided that X₁, X₂, and X₃ are not all N and at least one of X₁, X₂, or X₃ is N;

A1 is a bond, C(R₈)(R₉)—, —O—,—NR₈, —S—,—S(O)—, or —SO₂—;

A2 and Y are independently at each occurrence —C(R₈)(R₉)—, —O—, —NR₈, —S—, —S(O)—, or —SO₂—;

R₁ is H, halogen, —NR₈R₉, —P(O)(OR₈)(OR₉), —C(O)R₈, —C(O)NR₈R₉, —CN, C₁-C₆ alkyl, C₁-C₆ alkoxy, C₂-C₆ alkenyl, C₂-C₆ alkynyl, C₃-C₁₀ cycloalkyl, C₃-C₈ spirocycloalkyl, spiroheterocyclyl, heterocyclyl, aryl, or heteroaryl, wherein the alkyl, alkoxy, alkenyl, alkynyl, cycloalkyl, spirocycloalkyl, spiroheterocyclyl, heterocyclyl, aryl, or heteroaryl is optionally substituted with one or more R₆;

R₂ and R₃ are independently at each occurrence H, halogen, —OH, —NH₂, —CN, C1-C6 alkyl, C1-C6 alkoxy, C₂-C₆ alkenyl, or C₂-C₆ alkynyl, wherein the alkyl, alkoxy, alkenyl, or alkynyl is optionally substituted with one or more R₇;

R₄ is H, halogen, —OH, —NH₂, —CN, C₁-C₆ alkyl, C₁-C₆ alkoxy, C₂-C₆ alkenyl, or C₂-C₆ alkynyl, wherein the alkyl, alkoxy, alkenyl, or alkynyl is optionally substituted with one or more R₇,

or R₄ and R₉ when taken together can form C₃-C₁₀ cycloalkyl, heterocyclyl, aryl, or heteroaryl, wherein the cycloalkyl, heterocyclyl, aryl, or heteroaryl is optionally substituted with one or more R₁₀;

R₅ is H, halogen, —CN, —OR₈, —NR₈R₉, —C(O)R₈, —C(O)OR₈, —C(O)NR₈R₉, —NR₈C(O)R₉, —S(O)R₈, —S(O)₂R₈, —NR₈S(O)₂R₉, —S(O)₂NR₈R₉, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein alkyl, alkenyl, alkynyl, cycloalkyl, heterocyclyl, aryl, or heteroaryl is optionally substituted with one or more R₇;

R₆ is independently at each occurrence oxo, halogen, —CN, OH, —NR₈R₉, —OR₈, —C(O)R₈, —C(O)OR₈, —C(O)NR₈R₉, —NR₈C(O)R₉, —S(O)R₈, —S(O)₂R₈, —NR₈S(O)₂R₉, —S(O)₂NR₈R₉, C₁-C₆ alkyl, C₂-C₆ alkenyl, C₂-C₆ alkynyl, C₃-C₈ cycloalkyl, heterocyclyl, aryl, or heteroaryl, wherein each alkyl, alkenyl, alkynyl, cycloalkyl, heterocyclyl, aryl, or heteroaryl is optionally substituted with one or more R₁₀; or

two R₆ can combine to form C₃-C₁₀ cycloalkyl, heterocyclyl, aryl, or heteroaryl, wherein the cycloalkyl, heterocyclyl, aryl, or heteroaryl is optionally substituted with one or more R₁₀;

R₇ is independently at each occurrence oxo, halogen, —CN, —OR₈, —C(O)R₈, —C(O)OR₈, —C(O)NR₈R₉, —NR₈C(O)R₉, —S(O)R₈, —S(O)₂R₈, —NR₈S(O)₂R₉, —S(O)₂NR₈R₉, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl;

R₈ is independently at each occurrence H, OH, halogen, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀;

R₉ is independently at each occurrence H, halogen, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀;

or R₈ and R₉ when taken together form a C₃-C₆ cycloalkyl or heterocyclyl, wherein the cycloalkyl or heterocyclyl is optionally substituted with R₁₀; and

Ro is independently at each occurrence oxo, halogen, —CN, —OR₁₁, —C(O)R₁₁, —C(O)OR₁₁, —C(O)NR₁₁R₁₂, —NR₁₁R₁₂, —NR₁₁C(O)R₁₂, —S(O)R₁₁, —S(O)₂R₁₁, —NR₁₁S(O)₂R₁₂, —S(O)₂NR₁₁R₁₂, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl; and

R₁₁ and R₁₂ are independently H, C₁-C₆ alkyl, C₁-C₆ haloalkyl, C₁-C₆ alkoxy, C₂-C₆ alkenyl, C₂-C₆ alkynyl, C₃-C₈ cycloalkyl, heterocyclyl, aryl, or heteroaryl.

In some embodiments, X₁ is N or C(R₅). In some embodiments, X₁ is N. In some embodiments, X₁ is C(R₅). In some embodiments, X₂ is N. In some embodiments, X₂ is C(R₅). In some embodiments, X₃ is N. In some embodiments, X₃ is C(R₅).

In some embodiments, A₁ is a bond, —C(R₈)(R₉)—, —O—, —NR₈—, —S—, —S(O)—, or —SO₂—.

In some embodiments, A₂ and Y are independently at each occurrence —C(R₈)(R₉)—, —O—, —NR₈—, or —SO₂—.

In some embodiments, A₁ is —C(R₈)(R₉)—, —O—, —NR₈—, —S—, —S(O)—, or —SO₂—. In some embodiments, A₁ is a bond. In some embodiments, A₁ is —C(R₈)(R₉)— or —O—. In some embodiments, A₁ is —C(R₈)(R₉)—. In some embodiments, A₁ is—O—. In some embodiments, A₁ is —NR₈—. In some embodiments, A₁ is —S—. In some embodiments, A₁ is —S(O)—. In some embodiments, A₁ is —SO₂—.

In some embodiments, A₂ is —C(R₈)(R₉)—, —O—, —NR₈—, or —SO₂—. In some embodiments, A₂ is —C(R₈)(R₉)— or —O—. In some embodiments, A₂ is —C(R₈)(R₉)—. In some embodiments, A₂ is —O—. In some embodiments, A₂ is —NR₈—. In some embodiments, A₂ is —S—. In some embodiments, A₂ is —S(O)—. In some embodiments, A₂ is —SO₂—.

In some embodiments, Y is —C(R₈)(R₉)—, —O—, —NR₈—, or —SO₂—. In some embodiments, Y is —C(R₈)(R₉)— or —O—. In some embodiments, Y is —C(R₈)(R₉)—. In some embodiments, Y is —O—. In some embodiments, Y is —NR₈—. In some embodiments, Y is —S—. In some embodiments, Y is —S(O)—. In some embodiments, Y is —SO₂—.

In some embodiments, R₁ is H, halogen, —NR₈R₉, —P(O)(OR₈)(OR₉), —C(O)R₈, —C(O)NR₈R₉, —CN, C₁-C₆ alkyl, C₁-C₆ alkoxy, C₂-C₆ alkenyl, C₂-C₆ alkynyl, C₃-C₁₀ cycloalkyl, C₃-C₈ spirocycloalkyl, spiroheterocyclyl, heterocyclyl, aryl, or heteroaryl. In some embodiments, R₁ is H. In some embodiments, R₁ is —NR₈R₉. In some embodiments, R₁ is —P(OR₈)(OR₉). In some embodiments, R₁ is —C(O)R₈. In some embodiments, R₁ is —C(O)NR₈R₉. In some embodiments, R₁ is —CN. In some embodiments, R₁ is C₁-C₆ alkyl. In some embodiments, R₁ is C₁-C₆ alkoxy. In some embodiments, R₁ is C₂-C₆ alkenyl. In some embodiments, R₁ is C₂-C₆ alkynyl. In some embodiments, R₁ is C₃-C₁₀ cycloalkyl. In some embodiments, R₁ is C₃-C₈ spirocycloalkyl. In some embodiments, R₁ is spiroheterocyclyl. In some embodiments, R₁ is heterocyclyl. In some embodiments, R₁ is aryl. In some embodiments, R₁ is heteroaryl.

In some embodiments, R₁ is C₁-C₆ alkyl is optionally substituted with one or more R₆. In some embodiments, R₁ is C₁-C₆ alkoxy is optionally substituted with one or more R₆. In some embodiments, R₁ is C₂-C₆ alkenyl is optionally substituted with one or more R₆. In some embodiments, R₁ is C₂-C₆ alkynyl is optionally substituted with one or more R₆. In some embodiments, R₁ is C₃-C₁₀ cycloalkyl is optionally substituted with one or more R₆. In some embodiments, R₁ is C₃-C₈ spirocycloalkyl is optionally substituted with one or more R₆. In some embodiments, R₁ is spiroheterocyclyl is optionally substituted with one or more R₆. In some embodiments, R₁ is heterocyclyl is optionally substituted with one or more R₆. In some embodiments, R₁ is aryl is optionally substituted with one or more R₆. In some embodiments, R₁ is heteroaryl is optionally substituted with one or more R₆.

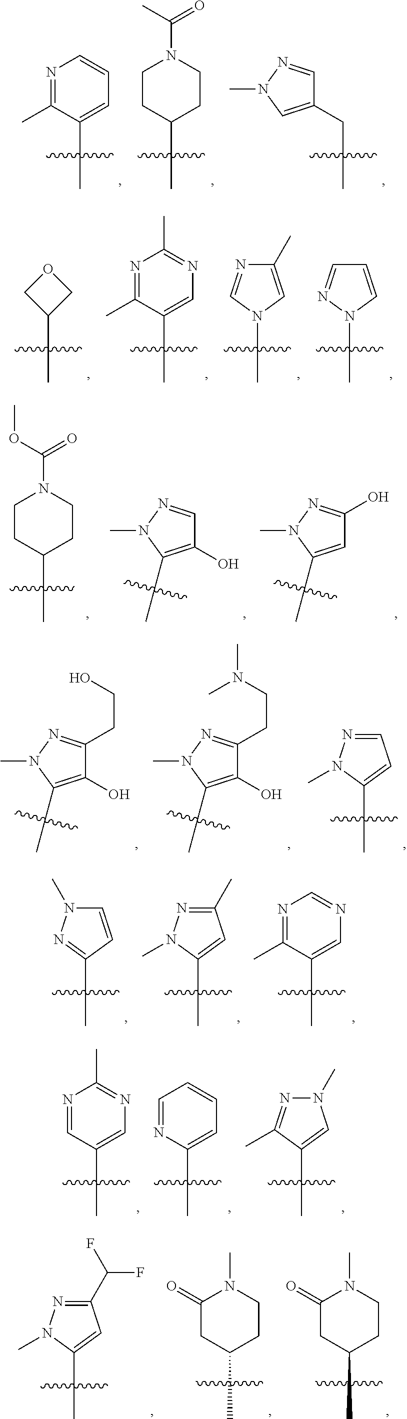

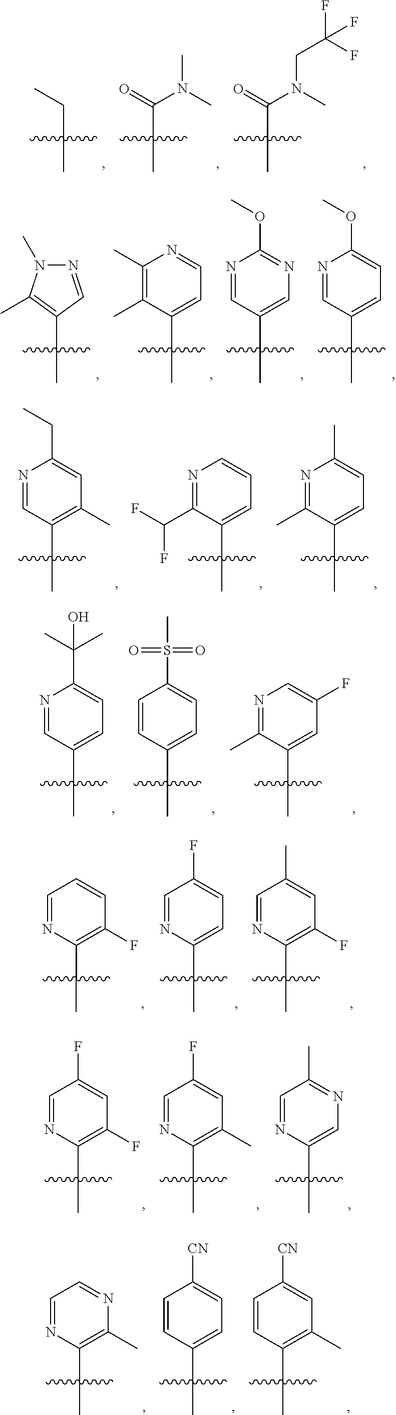

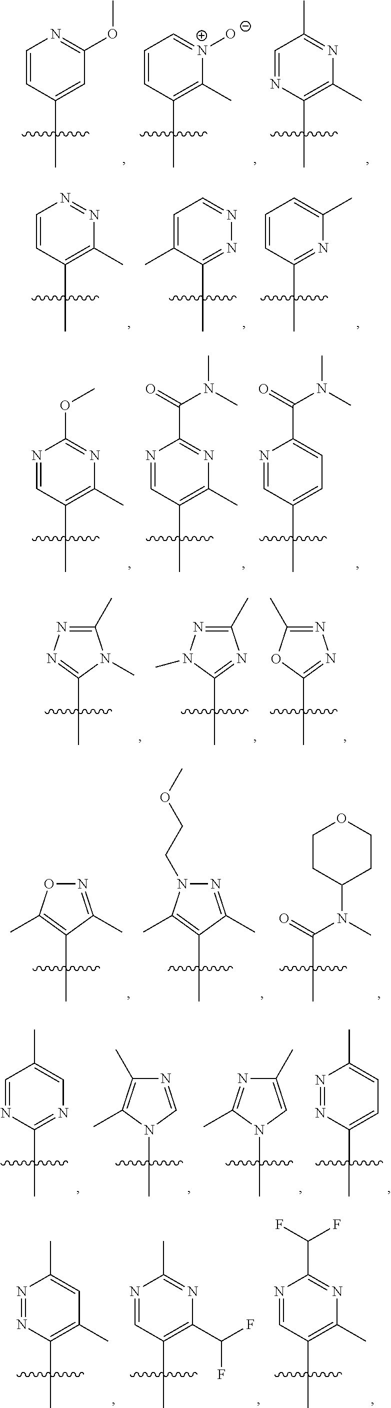

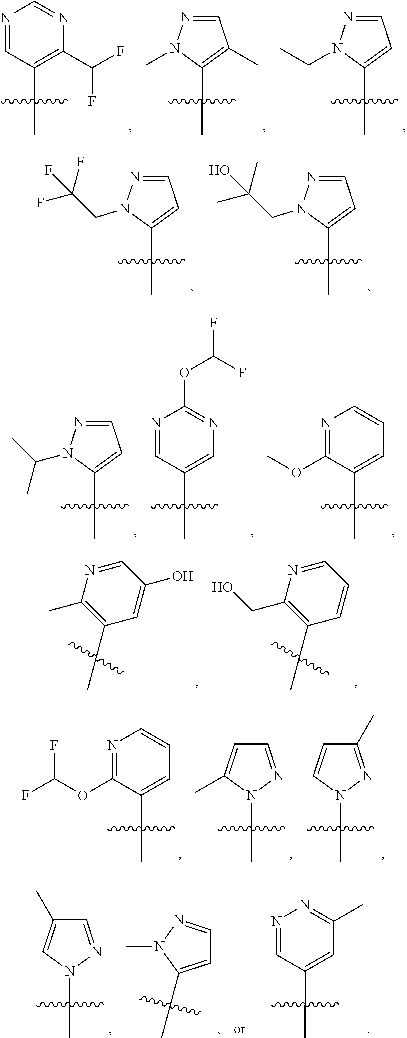

In another embodiment, R₁ is

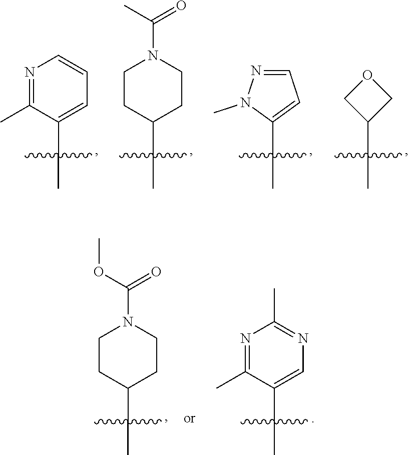

In another embodiment, R₁ is

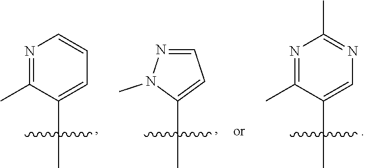

In another embodiment, R₁ is

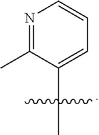 [SMILES: *c1cccnc1C]

In another embodiment, R₁ is

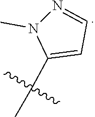 [SMILES: *c1ccnn1C]

In another embodiment, R₁ is

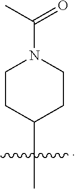 [SMILES: *C1CCN(C(C)=O)CC1]

In some embodiments, R₁ is

In some embodiments, R₂ is independently at each occurrence H, halogen, —OH, —NH₂, —CN, C₁-C₆ alkyl, C₁-C₆ alkoxy, C₂-C₆ alkenyl, or C₂-C₆ alkynyl. In some embodiments, R₂ is H. In some embodiments, R₂ is halogen. In some embodiments, R₂ is —OH. In some embodiments, R₂ is —NH₂. In some embodiments, R₂ is —CN. In some embodiments, R₂ is C₁-C₆ alkyl. In some embodiments, R₂ is C₁-C₆ alkoxy. In some embodiments, R₂ is C₂-C₆ alkenyl. In some embodiments, R₂ is C₂-C₆ alkynyl.

In some embodiments, R₂ is C₁-C₆ alkyl optionally substituted with one or more R₇. In some embodiments, R₂ is C₁-C₆ alkoxy optionally substituted with one or more R₇. In some embodiments, R₂ is C₂-C₆ alkenyl optionally substituted with one or more R₇. In some embodiments, R₂ is C₂-C₆ alkynyl optionally substituted with one or more R₇.

In some embodiments, R₃ is independently at each occurrence H, halogen, —OH, —NH₂, —CN, C₁-C₆ alkyl, C₁-C₆ alkoxy, C₂-C₆ alkenyl, or C₂-C₆ alkynyl. In some embodiments, R₃ is H. In some embodiments, R₃ is halogen. In some embodiments, R₃ is —OH. In some embodiments, R₃ is —NH₂. In some embodiments, R₃ is —CN. In some embodiments, R₃ is C₁-C₆ alkyl. In some embodiments, R₃ is C₁-C₆ alkoxy. In some embodiments, R₃ is C₂-C₆ alkenyl. In some embodiments, R₃ is C₂-C₆ alkynyl.

In some embodiments, R₃ is C₁-C₆ alkyl optionally substituted with one or more R₇. In some embodiments, R₃ is C₁-C₆ alkoxy optionally substituted with one or more R₇. In some embodiments, R₃ is C₂-C₆ alkenyl optionally substituted with one or more R₇. In some embodiments, R₃ is C₂-C₆ alkynyl optionally substituted with one or more R₇.

In some embodiments, Ra is independently at each occurrence H, halogen, —OH, —NH₂, —CN, C₁-C₆ alkyl, C₁-C₆ alkoxy, C₂-C₆ alkenyl, or C₂-C₆ alkynyl. In some embodiments, R₄ is H. In some embodiments, R₄ is halogen. In some embodiments, R₄ is —OH. In some embodiments, R+is —NH₂. In some embodiments, R₄ is —CN. In some embodiments, R₄ is C₁-C₆ alkyl. In some embodiments, R₄ is C₁-C₆ alkoxy. In some embodiments, R₄ is C₂-C₆ alkenyl. In some embodiments, R₄ is C₂-C₆ alkynyl.

In some embodiments, R₄ is C₁-C₆ alkyl optionally substituted with one or more R₇. In some embodiments, R₄ is C₁-C₆ alkoxy optionally substituted with one or more R₇. In some embodiments, R₄ is C₂-C₆ alkenyl optionally substituted with one or more R₇. In some embodiments, R₄ is C₂-C₆ alkynyl optionally substituted with one or more R₇.

In some embodiments, R₄ and R₉ can form C₃-C₁₀ cycloalkyl, heterocyclyl, aryl, or heteroaryl. In some embodiments, R₄ and R₉ can form C₃-C₁₀ cycloalkyl, heterocyclyl, aryl, or heteroaryl, wherein the cycloalkyl, heterocyclyl, aryl, or heteroaryl is optionally substituted with one or more R₁₀.

In some embodiments, R₄ and R₉ can form C₃-C₁₀ cycloalkyl. In some embodiments, R₄ and R₉ can form heterocyclyl. In some embodiments, R₄ and R₉ can form aryl. In some embodiments, R₄ and R₉ can form heteroaryl.

In some embodiments, R₄ and R₉ can form C₃-C₁₀ cycloalkyl optionally substituted with one or more R₁₀. In some embodiments, R₄ and R₉ can form heterocyclyl optionally substituted with one or more R₁₀. In some embodiments, R₄ and R₉ can form aryl optionally substituted with one or more R₁₀. In some embodiments, R₄ and R₉ can form heteroaryl optionally substituted with one or more R₁₀.

In some embodiments, R₅ is H, halogen, —CN, —OR₈, —NR₈R₉, —C(O)R₈, —C(O)OR₈, —C(O)NR₈R₉, —NR₈C(O)R₉, —S(O)R₈, —S(O)₂R₈, —NR₈S(O)₂R₉, —S(O)₂NR₈R₉, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl, wherein alkyl, alkenyl, alkynyl, cycloalkyl, heterocyclyl, aryl, or heteroaryl is optionally substituted with one or more R₇.

In some embodiments, R₅ is H, halogen, —CN, —OR₈, —NR₈R₉, —C(O)R₈, —C(O)OR₈, —C(O)NR₈R₉, —NR₈C(O)R₉, —S(O)R₈, —S(O)₂R₈, —NR₈S(O)₂R₉, —S(O)₂NR₈R₉, C₁-C₆ alkyl, C₁-C₆ haloalkyl, C₂-C₆ alkenyl, C₂-C₆ alkynyl, C₃-C₁₀ cycloalkyl, heterocyclyl, aryl, or heteroaryl. In some embodiments, R₅ is H. In some embodiments, R₅ is halogen. In some embodiments, R₅ is CN. In some embodiments, R₅ is —OR₈. In some embodiments, R₅ is —NR₈R₉. In some embodiments, R₅ is —C(O)R₈. In some embodiments, R₅ is —C(O)OR₈. In some embodiments, R₅ is —C(O)NR₈R₉. In some embodiments, R₅ is —NR₈C(O)R₉. In some embodiments, R₅ is —S(O)R₈. In some embodiments, R₅ is —S(O)₂R₈. In some embodiments, R₅ is —NR₈S(O)₂R₉. In some embodiments, R₅ is —S(O)₂NR₈R₉. In some embodiments, R₅ is C₁-C₆ alkyl. In some embodiments, R₅ is C₁-C₆ haloalkyl. In some embodiments, R₅ is C₂-C₆ alkenyl. In some embodiments, R₅ is C₂-C₆ alkynyl. In some embodiments, R₅ is C₃-C₁₀ cycloalkyl. In some embodiments, R₅ is heterocyclyl. In some embodiments, R₅ is aryl. In some embodiments, R₅ is heteroaryl.

In some embodiments, R₅ is C₁-C₆ alkyl, C₁-C₆ haloalkyl, C₂-C₆ alkenyl, or C₂-C₆ alkynyl.

In some embodiments, R₅ is C₁-C₆ alkyl. In some embodiments, R₅ is methyl. In some embodiments, R₅ is ethyl. In some embodiments, R₅ is propyl. In some embodiments, R₅ is butyl. In some embodiments, R₅ is pentyl. In some embodiments, R₅ is hexyl.

In some embodiments, R₅ is C₁-C₆ alkyl optionally substituted with one or more R₇. In some embodiments, R₅ is methyl optionally substituted with one or more R₇. In some embodiments, R₅ is ethyl optionally substituted with one or more R₇. In some embodiments, R₅ is propyl optionally substituted with one or more R₇. In some embodiments, R₅ is butyl optionally substituted with one or more R₇. In some embodiments, R₅ is pentyl optionally substituted with one or more R₇. In some embodiments, R₅ is hexyl optionally substituted with one or more R₇.

In some embodiments, R₅ is C₁-C₆ haloalkyl. In some embodiments, R₅ is halomethyl. In some embodiments, R₅ is haloethyl. In some embodiments, R₅ is halopropyl. In some embodiments, R₅ is halobutyl. In some embodiments, R₅ is halopentyl. In some embodiments, R₅ is halohexyl.

In some embodiments, R₅ is C₂-C₆ alkenyl. In some embodiments, R₅ is C₂-C₆ alkynyl.

In some embodiments, R₅ is C₂-C₆ alkenyl optionally substituted with one or more R₇. In some embodiments, R₅ is C₂-C₆ alkynyl optionally substituted with one or more R₇.

In some embodiments, R₅ is C₃-C₁₀ cycloalkyl.

In some embodiments, R₅ is C₃-C₁₀ cycloalkyl, wherein the cycloalkyl is optionally substituted with one or more R₇.

In some embodiments, R₅ is C₃-C₁₀ cycloalkyl. In some embodiments, R₅ is monocylic C₃-C₁₀ cycloalkyl. In some embodiments, R₅ is bicyclic C₃-C₁₀ cycloalkyl. In some embodiments, R₅ is polycyclic C₃-C₁₀ cycloalkyl.

In some embodiments, R₅ is C₃-C₁₀ cycloalkyl optionally substituted with one or more R₇. In some embodiments, R₅ is monocylic C₃-C₁₀ cycloalkyl optionally substituted with one or more R₇. In some embodiments, R₅ is bicyclic C₃-C₁₀ cycloalkyl optionally substituted with one or more R₇. In some embodiments, R₅ is polycyclic C₃-C₁₀ cycloalkyl optionally substituted with one or more R₇.

In some embodiments, R₅ is heterocyclyl, aryl, or heteroaryl. In some embodiments, R₅ is heterocyclyl. In some embodiments, R₅ is aryl. In some embodiments, R₅ is phenyl.

In some embodiments, R₅ is heterocyclyl, aryl, or heteroaryl, wherein the heterocyclyl, aryl, or heteroaryl is optionally substituted with one or more R₇. In some embodiments, R₅ is heterocyclyl optionally substituted with one or more R₇. In some embodiments, R₅ is aryl optionally substituted with one or more R₇. In some embodiments, R₅ is phenyl optionally substituted with one or more R₇.

In some embodiments, R₅ is heteroaryl. In some embodiments, R₅ is pyridine. In some embodiments, R₅ is imidazolyl. In some embodiments, R₅ is pyrazolyl. In some embodiments, R₅ is pyrimidinyl.

In some embodiments, R₅ is heteroaryl optionally substituted with one or more R₇. In some embodiments, R₅ is pyridine optionally substituted with one or more R₇. In some embodiments, R₅ is imidazolyl optionally substituted with one or more R₇. In some embodiments, R₅ is pyrazolyl optionally substituted with one or more R₇. In some embodiments, R₅ is pyrimidinyl optionally substituted with one or more R₇.

In some embodiments, R₅ is —CF₃. In some embodiments, R₅ is —CHF₂. In some embodiments, R₅ is —CH₂F.

In some embodiments, R₆ is independently at each occurrence oxo, halogen, —CN, OH, —NR₈R₉, —OR₈, —C(O)R₈, —C(O)OR₈, —C(O)NR₈R₉, —NR₈C(O)R₉, —S(O)R₈, —S(O)₂R₈, —NR₈S(O)₂R₉, —S(O)₂NR₈R₉, C₁-C₆ alkyl, C₂-C₆ alkenyl, C₂-C₆ alkynyl, C₃-C₈ cycloalkyl, heterocyclyl, aryl, or heteroaryl. In some embodiments, R₆ is oxo. In some embodiments, R₆ is halogen. In some embodiments, R₆ is CN. In some embodiments, R₆ is OH. In some embodiments, R₆ is —NR₈R₉. In some embodiments, R₆ is —OR₈. In some embodiments, R₆ is —NR₈R₉. In some embodiments, R₆ is —C(O)R₈. In some embodiments, R₆ is —C(O)OR₈. In some embodiments, R₆ is —C(O)NR₈R₉. In some embodiments, R₆ is —NR₈C(O)R₉. In some embodiments, R₆ is —S(O)R₈. In some embodiments, R₆ is —S(O)₂R₈. In some embodiments, R₆ is —NR₈S(O)₂R₉. In some embodiments, R₆ is —S(O)₂NR₈R₉. In some embodiments, R₆ is C₁-C₆ alkyl. In some embodiments, R₆ is C₁-C₆ haloalkyl. In some embodiments, R₆ is C₂-C₆ alkenyl. In some embodiments, R₆ is C₂-C₆ alkynyl. In some embodiments, R₆ is C₃-C₁₀ cycloalkyl. In some embodiments, R₆ is heterocyclyl. In some embodiments, R₆ is aryl. In some embodiments, R₆ is heteroaryl.

In some embodiments, R₆ is C₁-C₆ alkyl optionally substituted with one or more R₁₀. In some embodiments, R₆ is C₁-C₆ haloalkyl optionally substituted with one or more R₁₀. In some embodiments, R₆ is C₂-C₆ alkenyl optionally substituted with one or more R₁₀. In some embodiments, R₆ is C₂-C₆ alkynyl optionally substituted with one or more R₁₀. In some embodiments, R₆ is C₃-C₁₀ cycloalkyl optionally substituted with one or more R₁₀. In some embodiments, R₆ is heterocyclyl optionally substituted with one or more R₁₀. In some embodiments, R₆ is aryl optionally substituted with one or more R₁₀. In some embodiments, R₆ is heteroaryl optionally substituted with one or more R₁₀.

In another embodiment, two R₆ may combine to form C₃-C₁₀ cycloalkyl, heterocyclyl, aryl, or heteroaryl. In another embodiment, two R₆ may combine to form C₃-C₁₀ cycloalkyl. In another embodiment, two R₆ may combine to form a heteroaryl. In another embodiment, two R₆ may combine to form a heterocyclyl. In another embodiment, two R₆ may combine to form an aryl. In another embodiment, two R₆ may combine to form C₃-C₁₀ cycloalkyl, wherein the cycloalkyl is optionally substituted with one or more R₁₀. In another embodiment, two R₆ may combine to form a heteroaryl, wherein the heteroaryl is optionally substituted with one or more R₁₀. In another embodiment, two R₆ may combine to form a heterocyclyl, wherein the heterocyclyl is optionally substituted with one or more R₁₀. In another embodiment, two R₆ may combine to form an aryl wherein the aryl is optionally substituted with one or more R₁₀.

In some embodiments, R₇ is independently at each occurrence oxo, halogen, —CN, OR₈, —C(O)R₈, —C(O)OR₈, —C(O)NR₈R₉, —NR₈C(O)R₉, —S(O)R₈, —S(O)₂R₈, —NR₈S(O)₂R₉, —S(O)₂NR₈R₉, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₇ is independently at each occurrence oxo, halogen, or —CN. In some embodiments, R₇ is oxo. In some embodiments, R₇ is halogen. In some embodiments, R₇ is F, Cl, Br, or I. In some embodiments, R₇ is F or Cl. In some embodiments, R₇ is F. In some embodiments, R₇ is Cl. In some embodiments, R₇ is —CN.

In some embodiments, R₇ is independently at each occurrence —OR₈, —C(O)R₈, —C(O)OR₈, —C(O)NR₈R₉, —NR₈C(O)R₉, —S(O)R₈, —S(O)₂R₈, —NR₈S(O)₂R₉, or —S(O)₂NR₈R₉. In some embodiments, R₇ is —OR₈. In some embodiments, R₇ is —C(O)R₈. In some embodiments, R₇ is —C(O)OR₈. In some embodiments, R₇ is —C(O)NR₈R₉. In some embodiments, R₇ is —NR₈C(O)R₉. In some embodiments, R₇ is —S(O)R₈. In some embodiments, R₇ is —S(O)₂R₈. In some embodiments, R₇ is —NR₈S(O)₂R₉. In some embodiments, R₇ is —S(O)₂NR₈R₉.

In some embodiments, R₇ is independently at each occurrence C₁-C₆ alkyl, C₁-C₆ haloalkyl, C₂-C₆ alkenyl, or C₂-C₆ alkynyl.

In some embodiments, R₇ is C₁-C₆ alkyl. In some embodiments, R₇ is methyl. In some embodiments, R₇ is ethyl. In some embodiments, R₇ is propyl. In some embodiments, R₇ is butyl. In some embodiments, R₇is pentyl. In some embodiments, R₇ is hexyl.

In some embodiments, R₇ is C₁-C₆ haloalkyl. In some embodiments, R₇ is halomethyl. In some embodiments, R₇ is haloethyl. In some embodiments, R₇ is halopropyl. In some embodiments, R₇ is halobutyl. In some embodiments, R₇ is halopentyl. In some embodiments, R₇ is halohexyl.

In some embodiments, R₇ is C₂-C₆ alkenyl. In some embodiments, R₇ is C₂-C₆ alkynyl.

In some embodiments, R₇ is independently at each occurrence C₃-C₈ cycloalkyl or heterocyclyl. In some embodiments, R₇ is C₃-C₈ cycloalkyl. In some embodiments, R₇ is heterocyclyl.

In some embodiments, R₇ is independently at each occurrence aryl or heteroaryl. In some embodiments, R₇ is aryl. In some embodiments, R₇ is heteroaryl.

In some embodiments, R₈ is independently at each occurrence H, OH, halogen, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀.

In some embodiments, R₉ is independently at each occurrence H, halogen, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀.

In some embodiments, R₈ and R₉ are independently at each occurrence H, C₁-C₆ alkyl, C₁-C₆ alkoxy, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₈ and R₉ are independently at each occurrence H.

In some embodiments, R₈ and R₉ are independently at each occurrence C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl.

In some embodiments, R₈ and R₉ are independently at each occurrence C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀.

In some embodiments, R₈ is independently at each occurrence H, halogen, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀.

In some embodiments, R₈ is independently at each occurrence H, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl.

In some embodiments, R₈ is H.

In some embodiments, R₈ is halogen.

In some embodiments, R₈ is independently at each occurrence C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl.

In some embodiments, R₈ is independently at each occurrence C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀.

In some embodiments, R₈ is independently at each occurrence C₁-C₆ alkyl, C₁-C₆ alkoxy, C₂-C₆ alkenyl or C₂-C₆ alkynyl.

In some embodiments, R₈ is independently at each occurrence C₁-C₆ alkyl, C₁-C₆ alkoxy, C₂-C₆ alkenyl or C₂-C₆ alkynyl, wherein each alkyl, alkoxy, alkenyl or alkynyl is optionally substituted with one or more R₁₀.

In some embodiments, R₈ is C₁-C₆ alkyl. In some embodiments, R₈ is methyl. In some embodiments, R₈ is ethyl. In some embodiments, R₈ is propyl. In some embodiments, R₈ is butyl. In some embodiments, R₈ is pentyl. In some embodiments, R₈ is hexyl.

In some embodiments, R₈ is C₁-C₆ alkyl optionally substituted with one or more R₁₀. In some embodiments, R₈ is methyl optionally substituted with one or more R₁₀. In some embodiments, R₈ is ethyl optionally substituted with one or more R₁₀. In some embodiments, R₈ is propyl optionally substituted with one or more R₁₀. In some embodiments, R₈ is butyl optionally substituted with one or more R₁₀. In some embodiments, R₈ is pentyl optionally substituted with one or more R₁₀. In some embodiments, R₈ is hexyl optionally substituted with one or more R₁₀.

In some embodiments, R₈ is C₁-C₆ alkoxy. In some embodiments, R₈ is methoxy. In some embodiments, R₈ is ethoxy. In some embodiments, R₈ is propoxy. In some embodiments, R₈ is butoxy. In some embodiments, R₈ is pentoxy. In some embodiments, R₈ is hexoxy.

In some embodiments, R₈ is C₁-C₆ alkoxy optionally substituted with one or more R₁₀. In some embodiments, R₈ is methoxy optionally substituted with one or more R₁₀. In some embodiments, R₈ is ethoxy optionally substituted with one or more R₁₀. In some embodiments, R₈ is propoxy optionally substituted with one or more R₁₀. In some embodiments, R₈ is butoxy optionally substituted with one or more R₁₀. In some embodiments, R₈ is pentoxy optionally substituted with one or more R₁₀. In some embodiments, R₈ is hexoxy optionally substituted with one or more R₁₀.

In some embodiments, R₈ is C₂-C₆ alkenyl. In some embodiments, R₈ is C₂-C₆ alkynyl.

In some embodiments, R₈ is C₂-C₆ alkenyl optionally substituted with one or more R₁₀. In some embodiments, R₈ is C₂-C₆ alkynyl optionally substituted with one or more R₁₀.

In some embodiments, R₈ is independently at each occurrence C₃-C₈ cycloalkyl, heterocyclyl, aryl, or heteroaryl.

In some embodiments, R₈ is independently at each occurrence C₃-C₈ cycloalkyl, heterocyclyl, aryl, or heteroaryl, wherein each C₃-C₈ cycloalkyl, heterocyclyl, aryl, or heteroaryl is optionally substituted with one or more R₁₀.

In some embodiments, R₈ is C₃-C₈ cycloalkyl. In some embodiments, R₈ is heterocyclyl. In some embodiments, R₈ is aryl. In some embodiments, R₈ is heteroaryl.

In some embodiments, R₈ is C₃-C₈ cycloalkyl optionally substituted with one or more R₁₀. In some embodiments, R₈ is heterocyclyl optionally substituted with one or more R₁₀. In some embodiments, R₈ is aryl optionally substituted with one or more R₁₀. In some embodiments, R₈ is heteroaryl optionally substituted with one or more R₁₀.

In some embodiments, R₉ is independently at each occurrence H, halogen, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀.

In some embodiments, R₉ is independently at each occurrence H, C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl.

In some embodiments, R₉ is H.

In some embodiments, R₉ is halogen.

In some embodiments, R₉ is independently at each occurrence C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl.

In some embodiments, R₉ is independently at each occurrence C₁-C₆ alkyl, C₁-C₆ alkoxy, C₂-C₆ alkenyl, C₂-C₆ alkynyl, C₃-C₈ cycloalkyl, heterocyclyl, aryl, or heteroaryl, wherein each alkyl, alkoxy, alkenyl, alkynyl, cycloalkyl, heterocyclyl, aryl, or heteroaryl is optionally substituted with one or more R₁₀.

In some embodiments, R₉ is independently at each occurrence C₁-C₆ alkyl, C₁-C₆ alkoxy, C₂-C₆ alkenyl or C₂-C₆ alkynyl.

In some embodiments, R₉ is independently at each occurrence C₁-C₆ alkyl, C₁-C₆ alkoxy, C₂-C₆ alkenyl or C₂-C₆ alkynyl, wherein each alkyl, alkoxy, alkenyl, or alkynyl is optionally substituted with one or more R₁₀.

In some embodiments, R₉ is C₁-C₆ alkyl. In some embodiments, R₉ is methyl. In some embodiments, Ro is ethyl. In some embodiments, R₉ is propyl. In some embodiments, R₉ is butyl. In some embodiments, R₉ is pentyl. In some embodiments, R₉ is hexyl.

In some embodiments, R₉ is C₁-C₆ alkyl optionally substituted with one or more R₁₀. In some embodiments, R₉ is methyl optionally substituted with one or more R₁₀. In some embodiments, Ro is ethyl optionally substituted with one or more R₁₀. In some embodiments, R₉ is propyl optionally substituted with one or more R₁₀. In some embodiments, R₉ is butyl optionally substituted with one or more R₁₀. In some embodiments, Ro is pentyl optionally substituted with one or more R₁₀. In some embodiments, R₉ is hexyl optionally substituted with one or more R₁₀.

In some embodiments, R₉ is C₁-C₆ alkoxy. In some embodiments, R₉ is methoxy. In some embodiments, R₉ is ethoxy. In some embodiments, R₉ is propoxy. In some embodiments, R₉ is butoxy. In some embodiments, R₉ is pentoxy. In some embodiments, R₉ is hexoxy.

In some embodiments, R₉ is C₁-C₆ alkoxy optionally substituted with one or more R₁₀. In some embodiments, R₉ is methoxy optionally substituted with one or more R₁₀. In some embodiments, R₉ is ethoxy optionally substituted with one or more R₁₀. In some embodiments, R₉ is propoxy optionally substituted with one or more R₁₀. In some embodiments, R₉ is butoxy optionally substituted with one or more R₁₀. In some embodiments, R₉ is pentoxy optionally substituted with one or more R₁₀. In some embodiments, R₉ is hexoxy optionally substituted with one or more R₁₀.

In some embodiments, R₉ is C₂-C₆ alkenyl. In some embodiments, R₉ is C₂-C₆ alkynyl.

In some embodiments, R₉ is C₂-C₆ alkenyl optionally substituted with one or more R₁₀. In some embodiments, R₉ is C₂-C₆ alkynyl optionally substituted with one or more R₁₀.

In some embodiments, R₉ is independently at each occurrence C₃-C₈ cycloalkyl, heterocyclyl, aryl, or heteroaryl.

In some embodiments, R₉ is independently at each occurrence C₃-C₈ cycloalkyl, heterocyclyl, aryl, or heteroaryl, wherein each cycloalkyl, heterocyclyl, aryl, or heteroaryl is optionally substituted with one or more R₁₀.

In some embodiments, R₉ is C₃-C₈ cycloalkyl. In some embodiments, R₉ is heterocyclyl. In some embodiments, R₉ is aryl. In some embodiments, R₉ is heteroaryl.

In some embodiments, R₉ is C₃-C₈ cycloalkyl optionally substituted with one or more R₁₀. In some embodiments, R₉ is heterocyclyl optionally substituted with one or more R₁₀. In some embodiments, R₉ is aryl optionally substituted with one or more R₁₀. In some embodiments, R₉ is heteroaryl optionally substituted with one or more R₁₀.

In some embodiments, R₈ and R₉ when taken together form a C₃-C₆ cycloalkyl or heterocycle, wherein the cycloalkyl or heterocycle is optionally substituted with R₁₀.

In some embodiments, R₈ and R₉ when taken together form a C₃-C₆ cycloalkyl, wherein the cycloalkyl is optionally substituted with R₁₀. In some embodiments, R₈ and R₉ when taken together form a C₃-C₆ cycloalkyl. In some embodiments, R₈ and R₉ when taken together form cyclopropyl, wherein the cyclopropyl is optionally substituted with R₁₀. In some embodiments, R₈ and R₉ when taken together form cyclopropyl.

In some embodiments, R₈ and R₉ when taken together form a heterocycle, wherein the heterocycle is optionally substituted with R₁₀. In some embodiments, R₈ and R₉ when taken together form a 4-membered heterocycle optionally substituted with R₁₀. In some embodiments, R₈ and R₉ when taken together form azetidinyl optionally substituted with R₁₀. In some embodiments, R₈ and R₉ when taken together form oxetanyl optionally substituted with R₁₀.

In some embodiments, R₁₀ is independently at each occurrence oxo, halogen, —CN, —OR₁₁, —C(O)R₁₁, —C(O)OR₁₁, —C(O)NR₁₁R₁₂, —NR₁₁C(O)R₁₂, —S(O)R₁₁, —S(O)₂R₁₁, —NR₁₁S(O)₂R₁₂, —S(O)₂NR₁₁R₁₂, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₀ is independently at each occurrence oxo, halogen, —CN, —OR₁₁, —C(O)R₁₁, —C(O)OR₁₁, —C(O)NR₁₁R₁₂, —NR₁₁R₁₂, —NR₁₁C(O)R₁₂, —S(O)R₁₁, —S(O)₂R₁₁, —NR₁₁S(O)₂R₁₂, —S(O)₂NR₁₁R₁₂, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₀ is independently at each occurrence oxo, halogen, or —CN. In some embodiments, R₁₀ is oxo. In some embodiments, R₁₀ is halogen. In some embodiments, R₁₀ is F, Cl, Br, or I. In some embodiments, R₁₀ is F or Cl. In some embodiments, R₁₀ is F. In some embodiments, R₁₀ is Cl. In some embodiments, R₁₀ is —CN.

In some embodiments, R₁₀ is independently at each occurrence —OR₁₁, —C(O)R₁₁, —C(O)OR₁₁, —C(O)NR₁₁R₁₂, —NR₁₁C(O)R₁₂, —S(O)R₁₁I, —S(O)₂R₁₁, —NR₁₁S(O)₂R₁₂, or —S(O)₂NR₁₁R₁₂. In some embodiments, R₁₀ is —OR₁₁. In some embodiments, R₁₀ is —C(O)R₁₁. In some embodiments, R₁₀ is —C(O)OR₁₁. In some embodiments, R₁₀ is —C(O)NR₁₁R₁₂. In some embodiments, R₁₀ is —NR₁₁C(O)R₁₂. In some embodiments, R₁₀ is —S(O)R₁₁. In some embodiments, R₁₀ is —S(O)₂R₁₁. In some embodiments, R₁₀ is —NR₁₁IS(O)₂R₁₂. In some embodiments, R₁₀ is —S(O)₂NR₁₁R₁₂.

In some embodiments, R₁₀ is independently at each occurrence C₁-C₆ alkyl, C₁-C₆ haloalkyl, C₂-C₆ alkenyl, or C₂-C₆ alkynyl.

In some embodiments, R₁₀ is C₁-C₆ alkyl. In some embodiments, R₁₀ is methyl. In some embodiments, R₁₀ is ethyl. In some embodiments, R₁₀ is propyl. In some embodiments, R₁₀ is butyl. In some embodiments, R₁₀ is pentyl. In some embodiments, R₁₀ is hexyl.

In some embodiments, R₁₀ is C₁-C₆ haloalkyl. In some embodiments, R₁₀ is halomethyl. In some embodiments, R₁₀ is haloethyl. In some embodiments, R₁₀ is halopropyl. In some embodiments, R₁₀ is halobutyl. In some embodiments, R₁₀ is halopentyl. In some embodiments, R₁₀ is halohexyl.

In some embodiments, R₁₀ is C₂-C₆ alkenyl. In some embodiments, R₁₀ is C₂-C₆ alkynyl.

In some embodiments, R₁₀ is independently at each occurrence C₃-C₈ cycloalkyl or heterocyclyl. In some embodiments, R₁₀ is C₃-C₈ cycloalkyl. In some embodiments, R₁₀ is heterocyclyl.

In some embodiments, R₁₀ is independently at each occurrence aryl or heteroaryl. In some embodiments, R₁₀ is aryl. In some embodiments, R₁₀ is heteroaryl.

In some embodiments, R₁₀ is —OH.

In some embodiments, R₁₁ and R₁₂ are independently H, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₁ and R₁₂ are independently H.

In some embodiments, R₁₁ and R₁₂ are independently C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₁ is H, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₁ is H.

In some embodiments, R₁₁ is C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₁ is C₁-C₆ alkyl, C₁-C₆ haloalkyl, C₂-C₆ alkenyl, or C₂-C₆ alkynyl.

In some embodiments, R₁₁ is C₁-C₆ alkyl. In some embodiments, R₁₁ is methyl. In some embodiments, R₁₁ is ethyl. In some embodiments, R₁₁ is propyl. In some embodiments, R₁₁ is butyl. In some embodiments, R₁₁ is pentyl. In some embodiments, R₁₁ is hexyl.

In some embodiments, R₁₁ is C₁-C₆ haloalkyl. In some embodiments, R₁₁ is halomethyl. In some embodiments, R₁₁ is haloethyl. In some embodiments, R₁₁ is halopropyl. In some embodiments, R₁₁ is halobutyl. In some embodiments, R₁₁ is halopentyl. In some embodiments, R₁₁ is halohexyl.

In some embodiments, R₁₁ is C₂-C₆ alkenyl. In some embodiments, R₁₁ is C₂-C₆ alkynyl.

In some embodiments, R₁₁ is C₃-C₈ cycloalkyl, heterocyclyl, aryl, or heteroaryl. In some embodiments, R₁₁ is C₃-C₈ cycloalkyl. In some embodiments, R₁₁ is heterocyclyl. In some embodiments, R₁₁ is aryl. In some embodiments, R₁₁ is heteroaryl.

In some embodiments, R₁₂ is H, C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₂ is H.

In some embodiments, R₁₂ is C₁-C₆ alkyl, C₁-C₆ haloalkyl, C₂-C₆ alkenyl, C₂-C₆ alkynyl, C₃-C₈ cycloalkyl, heterocyclyl, aryl, or heteroaryl.

In some embodiments, R₁₂ is C₁-C₆ alkyl, C₁-C₆ haloalkyl, C₂-C₆ alkenyl, or C₂-C₆ alkynyl.

In some embodiments, R₁₂ is C₁-C₆ alkyl. In some embodiments, R₁₂ is methyl. In some embodiments, R₁₂ is ethyl. In some embodiments, R₁₂ is propyl. In some embodiments, R₁₂ is butyl. In some embodiments, R₁₂ is pentyl. In some embodiments, R₁₂ is hexyl.

In some embodiments, R₁₂ is C₁-C₆ haloalkyl. In some embodiments, R₁₂ is halomethyl. In some embodiments, R₁₂ is haloethyl. In some embodiments, R₁₂ is halopropyl. In some embodiments, R₁₂ is halobutyl. In some embodiments, R₁₂ is halopentyl. In some embodiments, R₁₂ is halohexyl.

In some embodiments, R₁₂ is C₂-C₆ alkenyl. In some embodiments, R₁₂ is C₂-C₆ alkynyl.

In some embodiments, R₁₂ is C₃-C₈ cycloalkyl, heterocyclyl, aryl, or heteroaryl. In some embodiments, R₁₂ is C₃-C₈ cycloalkyl. In some embodiments, R₁₂ is heterocyclyl. In some embodiments, R₁₂ is aryl. In some embodiments, R₁₂ is heteroaryl.

In some embodiments, the compounds of the present disclosure are represented by compounds of Formula II:

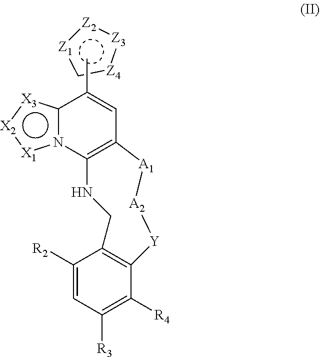

or pharmaceutically acceptable salts, prodrugs, solvates, hydrates, enantiomers, isomers, or tautomers thereof, wherein:

[symbol] represents optional double bonds which can form an aromatic when present; Z₁, Z₂, Z₃, and Z₄ are independently C, N, S, O, N(R₁₀), or C(R₁₀); and X₁, X₂, X₃, A₁, A₂, Y, R₂, R₃, R₄, R₁₀ are described as herein.

In some embodiments, the compounds of the present disclosure are represented by compounds of Formula III:

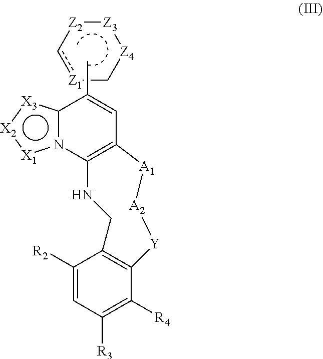

or pharmaceutically acceptable salts, prodrugs, solvates, hydrates, enantiomers, isomers, or tautomers thereof,
wherein:

[symbol] straight or curved represents optional double bonds that form a partially unsaturated ring or an aromatic ring when present;

Z₁, Z₂, Z₃, and Z₄ are independently C, N, O, N(R₁₀), or C(R₁₀), provided Z₁, Z₂, Z₃, and Z₄ are not all N or N(R₁₀) when [symbol] is present and aromatic; provided that no three N or N(R₁₀) are adjacent; provided that Z₁, Z₂, Z₃, and Z₄ are not O when [symbol] is present and aromatic; and X₁, X₂, X₃, A₁, A₂, Y, R₂, R₃, R₄, R₁₀ are described as herein.

In some embodiments, the compound is of formula Ia:

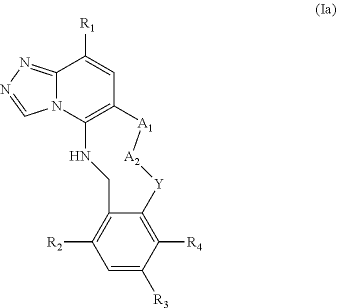

or a pharmaceutically acceptable salt, prodrug, solvate, hydrate, enantiomer, isomer, or tautomer thereof.

In some embodiments, the compound is of formula Ib:

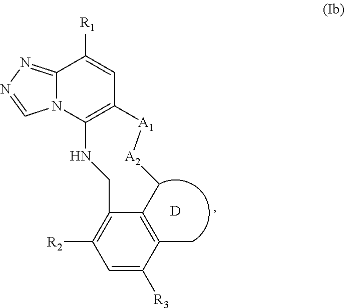

or a pharmaceutically acceptable salt, prodrug, solvate, hydrate, enantiomer, isomer, or tautomer thereof, wherein the D ring represents a C₃-C₁₀ cycloalkyl, heterocyclyl, aryl, or heteroaryl.

In some embodiments, the compound is of formula Ic:

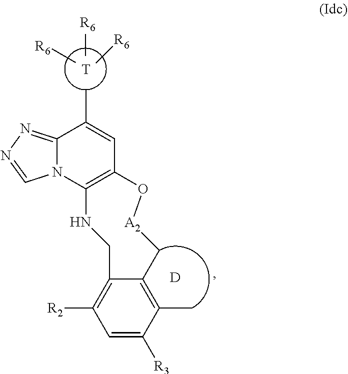

or a pharmaceutically acceptable salt, prodrug, solvate, hydrate, enantiomer, isomer, or tautomer thereof, wherein the D ring represents a C₃-C₁₀ cycloalkyl, heterocyclyl, aryl, or heteroaryl; and the T ring represents a C₃-C₁₀ cycloalkyl, C₃-C₈ spirocycloalkyl, spiroheterocyclyl, heterocyclyl, aryl, or heteroaryl.

In some embodiments, the compound is of formula Id:

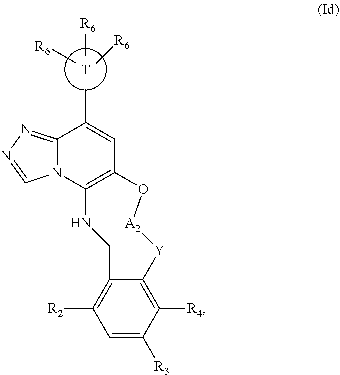

or a pharmaceutically acceptable salt, prodrug, solvate, hydrate, enantiomer, isomer, or tautomer thereof, wherein the T ring represents a C₃-C₁₀ cycloalkyl, C₃-C₈ spirocycloalkyl, spiroheterocyclyl, heterocyclyl, aryl, or heteroaryl.

In some embodiments, the compound is of formula Ie:

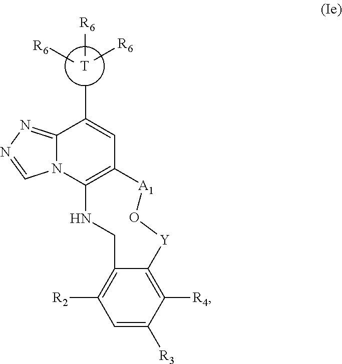

or a pharmaceutically acceptable salt, prodrug, solvate, hydrate, enantiomer, isomer, or tautomer thereof, wherein the T ring represents a C₃-C₁₀ cycloalkyl, C₃-C₈ spirocycloalkyl, spiroheterocyclyl, heterocyclyl, aryl, or heteroaryl.

In some embodiments, the compound is of formula If;

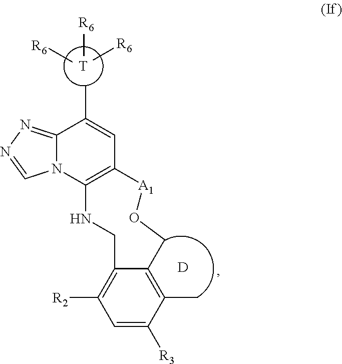

or a pharmaceutically acceptable salt, prodrug, solvate, hydrate, enantiomer, isomer, or tautomer thereof, wherein the D ring represents a C₃-C₁₀ cycloalkyl, heterocyclyl, aryl, or heteroaryl; and the T ring represents a C₃-C₁₀ cycloalkyl, C₃-C₈ spirocycloalkyl, spiroheterocyclyl, heterocyclyl, aryl, or heteroaryl.

In some embodiments, the compound is of formula Ig:

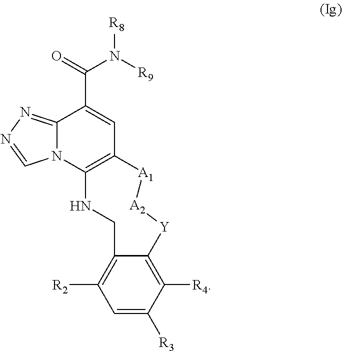

or a pharmaceutically acceptable salt, prodrug, solvate, hydrate, enantiomer, isomer, or tautomer thereof.

In some embodiments, the compound is of formula Ih:

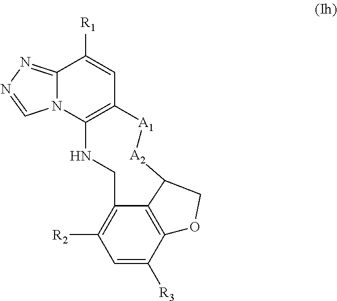

or a pharmaceutically acceptable salt, prodrug, solvate, hydrate, enantiomer, isomer, or tautomer thereof.

In some embodiments, the compound is of formula Ih-a:

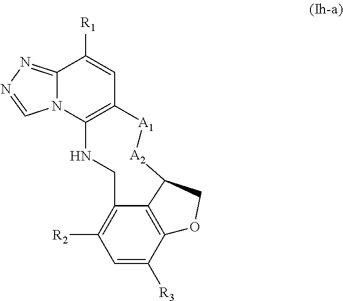

or a pharmaceutically acceptable salt, prodrug, solvate, hydrate, enantiomer, isomer, or tautomer thereof.

In some embodiments, the compound is of formula Ii:

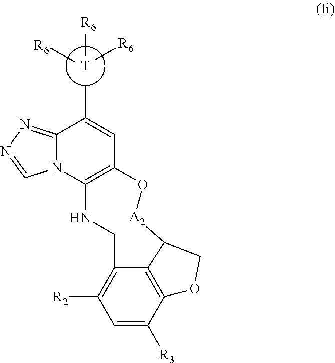

or a pharmaceutically acceptable salt, prodrug, solvate, hydrate, enantiomer, isomer, or tautomer thereof, wherein the T ring represents a C₃-C₁₀ cycloalkyl, C₃-C₈ spirocycloalkyl, spiroheterocyclyl, heterocyclyl, aryl, or heteroaryl.

In some embodiments, the compound is of formula Ii-a:

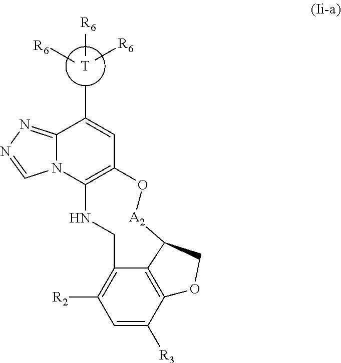

or a pharmaceutically acceptable salt, prodrug, solvate, hydrate, enantiomer, isomer, or tautomer thereof, wherein the T ring represents a C₃-C₁₀ cycloalkyl, C₃-C₈ spirocycloalkyl, spiroheterocyclyl, heterocyclyl, aryl, or heteroaryl.

In some embodiments, the compound is of formula Ii or a pharmaceutically acceptable salt, prodrug, solvate, hydrate, enantiomer, isomer, or tautomer thereof, wherein the T ring represents a heterocyclyl.

In some embodiments, the compound is of formula Ii-a or a pharmaceutically acceptable salt, prodrug, solvate, hydrate, enantiomer, isomer, or tautomer thereof, wherein the T ring represents a heterocyclyl.

In some embodiments, the compound is of formula Ij:

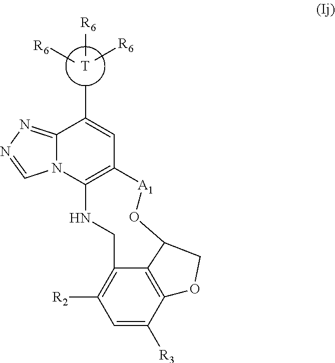

or a pharmaceutically acceptable salt, prodrug, solvate, hydrate, enantiomer, isomer, or tautomer thereof, wherein the T ring represents a C₃-C₁₀ cycloalkyl, C₃-C₈ spirocycloalkyl, spiroheterocyclyl, heterocyclyl, aryl, or heteroaryl.

In some embodiments, the compound is of formula Ij or a pharmaceutically acceptable salt, prodrug, solvate, hydrate, enantiomer, isomer, or tautomer thereof, wherein the T ring represents a heterocyclyl.

In some embodiments, the compound is of formula Ik:

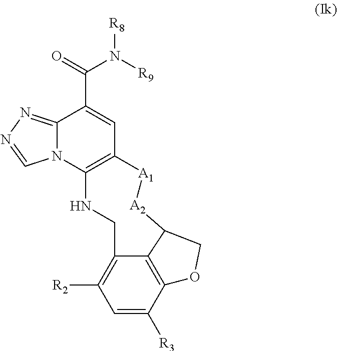

or a pharmaceutically acceptable salt, prodrug, solvate, hydrate, enantiomer, isomer, or tautomer thereof.

In some embodiments, the compound is of formula Ik-a:

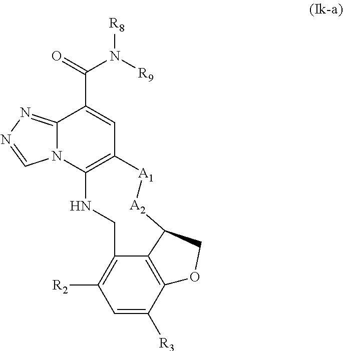

or a pharmaceutically acceptable salt, prodrug, solvate, hydrate, enantiomer, isomer, or tautomer thereof.

In some embodiments, inhibitors of the disclosure are and pharmaceutically acceptable salts, prodrugs, solvates, hydrates, enantiomers, isomers, and tautomers thereof are described in Table 1.

TABLE 1

| Compound No. | Compound Name |
|---|---|
| 1 | (15R)-10-(2-methyl-3-pyridyl)-13,17-dioxa-3,5,7,8-tetrazapentacyclo |
| | [13.6.1.04,12.05,9.018,22] docosa-1(22),4(12),6,8,10,18,20-heptaene |
| 3 | (S)-1-(4-(7a,8,13,14-tetrahydro-7H-[1,2,4]triazolo[4′,3′:1,6]pyrido[3,2- |
| | b]benzofuro[4,3-fg][1,4]oxazonin-4-yl)piperidin-1-yl)ethan-1-one |
| 4 | 1-(4-(12-fluoro-7,8,13,14-tetrahydro-[1,2,4]triazolo[4′,3′:1,6]pyrido[3,2- |
| | b]benzo[f][1,4]oxazonin-4-yl)piperidin-1-yl)ethan-1-one |
| 5 | 12-fluoro-4-(2-methylpyridin-3-yl)-7,8,13,14-tetrahydro- |
| | [1,2,4]triazolo[4′,3′:1,6]pyrido[3,2-b]benzo[f][1,4]oxazonine |
| 6 | 12-fluoro-4-(2-methylpyridin-3-yl)-6,8,13,14-tetrahydro- |
| | [1,2,4]triazolo[4′,3′:1,6]pyrido[3,2-c]benzo[g][1,5]oxazonine |
| 7 | (S)-4-((1-methyl-1H-pyrazol-4-yl)methyl)-7a,8,13,14-tetrahydro-7H- |
| | [1,2,4]triazolo[4′,3′:1,6]pyrido[3,2-b]benzofuro[4,3-fg][1,4]oxazonine |
| 8 | (S)-12-fluoro-4-(2-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 9 | (S)-1-(4-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)piperidin-1-yl)ethan-1-one |
| 10 | (S)-12-fluoro-4-((1-methyl-1H-pyrazol-4-yl)methyl)-7a,8,13,14-tetrahydro-7H- |
| | [1,2,4]triazolo[4′,3′:1,6]pyrido[3,2-b]benzofuro[4,3-fg][1,4]oxazonine |
| 11 | (S)-7a,8,13,14-tetrahydro-7H-[1,2,4]triazolo[4′,3′:1,6]pyrido[3,2- |
| | b]benzofuro[4,3-fg][1,4]oxazonine |
| 12 | (S)-12-fluoro-4-(oxetan-3-yl)-7a,8,13,14-tetrahydro-7H- |
| | [1,2,4]triazolo[4′,3′:1,6]pyrido[3,2-b]benzofuro[4,3-fg][1,4]oxazonine |
| 13 | (S)-4-(2,4-dimethylpyrimidin-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 14 | 4-(2,4-dimethylpyrimidin-5-yl)-12-fluoro-7,8,13,14-tetrahydro- |
| | [1,2,4]triazolo[4′,3′:1,6]pyrido[3,2-b]benzo[f][1,4]oxazonine |
| 15 | (S)-12-fluoro-4-(4-methyl-1H-imidazol-1-yl)-7a,8,13,14-tetrahydro-7H- |
| | [1,2,4]triazolo[4′,3′:1,6]pyrido[3,2-b]benzofuro[4,3-fg][1,4]oxazonine |
| 16 | methyl (S)-4-(12-fluoro-7a,8,13,14-tetrahydro-7H- |
| | [1,2,4]triazolo[4′,3′:1,6]pyrido[3,2-b]benzofuro[4,3-fg][1,4]oxazonin-4- |
| | yl)piperidine-1-carboxylate |
| 17 | (S)-12-fluoro-4-(1-methyl-1H-pyrazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 18 | (S)-4-(1,3-dimethyl-1H-pyrazol-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 19 | (S)-12-fluoro-4-(4-methylpyrimidin-5-yl)-7a,8,13,14-tetrahydro-7H- |
| | [1,2,4]triazolo[4′,3′:1,6]pyrido[3,2-b]benzofuro[4,3-fg][1,4]oxazonine |
| 20 | (S)-12-fluoro-4-(2-methylpyrimidin-5-yl)-7a,8,13,14-tetrahydro-7H- |
| | [1,2,4]triazolo[4′,3′:1,6]pyrido[3,2-b]benzofuro[4,3-fg][1,4]oxazonine |
| 21 | (S)-12-fluoro-4-(pyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 22 | (S)-4-(1,3-dimethyl-1H-pyrazol-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 23 | (S)-4-(3-(difluoromethyl)-1-methyl-1H-pyrazol-4-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 24 | (S)-4-((S)-12-fluoro-7a,8,13,14-tetrahydro-7H- |
| | [1,2,4]triazolo[4′,3′:1,6]pyrido[3,2-b]benzofuro[4,3-fg][1,4]oxazonin-4-yl)-1- |
| | methylpiperidin-2-one |
| 25 | (R)-4-((S)-12-fluoro-7a,8,13,14-tetrahydro-7H- |
| | [1,2,4]triazolo[4′,3′:1,6]pyrido[3,2-b]benzofuro[4,3-fg][1,4]oxazonin-4-yl)-1- |
| | methylpiperidin-2-one |
| 26 | (S)-4-ethyl-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 27 | (S)-12-fluoro-4-(1H-pyrazol-1-yl)-7a,8,13,14-tetrahydro-7H- |
| | [1,2,4]triazolo[4′,3′:1,6]pyrido[3,2-b]benzofuro[4,3-fg][1,4]oxazonine |
| 28 | (S)-4-(1,5-dimethyl-1H-pyrazol-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 29 | (S)-4-(2,3-dimethylpyridin-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 30 | (S)-12-fluoro-4-(2-methoxypyrimidin-5-yl)-7a,8,13,14-tetrahydro-7H- |
| | [1,2,4]triazolo[4′,3′:1,6]pyrido[3,2-b]benzofuro[4,3-fg][1,4]oxazonine |
| 31 | (S)-12-fluoro-4-(6-methoxypyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 32 | (S)-4-(6-ethyl-4-methylpyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 33 | (S)-4-(2-(difluoromethyl)pyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 34 | (S)-4-(2,6-dimethylpyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 35 | (S)-2-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)pyridin-2-yl)propan-2-ol |
| 36 | (S)-12-fluoro-4-(4-(methylsulfonyl)phenyl)-7a,8,13,14-tetrahydro-7H- |
| | [1,2,4]triazolo[4′,3′:1,6]pyrido[3,2-b]benzofuro[4,3-fg][1,4]oxazonine |
| 37 | (S)-12-fluoro-N,N-dimethyl-7a,8,13,14-tetrahydro-7H- |
| | [1,2,4]triazolo[4′,3′:1,6]pyrido[3,2-b]benzofuro[4,3-fg][1,4]oxazonine-4- |
| | carboxamide |
| 38 | (S)-12-fluoro-N-methyl-N-(2,2,2-trifluoroethyl)-7a,8,13,14-tetrahydro-7H- |
| | [1,2,4]triazolo[4′,3′:1,6]pyrido[3,2-b]benzofuro[4,3-fg][1,4]oxazonine-4- |
| | carboxamide |
| 39 | (S)-12-fluoro-4-(1-methyl-1H-pyrazol-3-yl)-7a,8,13,14-tetrahydro-7H- |
| | [1,2,4]triazolo[4′,3′:1,6]pyrido[3,2-b]benzofuro[4,3-fg][1,4]oxazonine |
| 40 | (S)-12-fluoro-4-(5-fluoro-2-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 41 | (S)-12-fluoro-4-(3-fluoropyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 42 | (S)-12-fluoro-4-(5-fluoropyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 43 | (S)-12-fluoro-4-(3-fluoro-5-methylpyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 44 | (S)-4-(3,5-difluoropyridin-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 45 | (S)-12-fluoro-4-(5-fluoro-3-methylpyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 46 | (S)-12-fluoro-4-(5-methylpyrazin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 47 | (S)-12-fluoro-4-(3-methylpyrazin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 48 | (S)-4-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)benzonitrile |
| 49 | (S)-4-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-3-methylbenzonitrile |
| 50 | (S)-12-fluoro-4-(2-methoxypyridin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 51 | (S)-3-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-2-methylpyridine 1-oxide |
| 52 | (S)-4-(3,5-dimethylpyrazin-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 53 | (S)-12-fluoro-4-(3-methylpyridazin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 54 | (S)-12-fluoro-4-(4-methylpyridazin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 55 | (S)-12-fluoro-4-(6-methylpyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 56 | (S)-12-fluoro-4-(2-methylpyrimidin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 57 | (S)-12-fluoro-4-(2-methoxy-4-methylpyrimidin-5-yl)-7a,8,13,14-tetrahydro-7H- |
| | [1,2,4]triazolo[4′,3′:1,6]pyrido[3,2-b]benzofuro[4,3-fg][1,4]oxazonine |
| 58 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-N,N,4-trimethylpyrimidine-2- |
| | carboxamide |
| 59 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-N,N-dimethylpicolinamide |
| 60 | (S)-4-(1,3-dimethyl-1H-1,2,4-triazol-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 61 | (S)-12-fluoro-4-(5-methyl-1,3,4-oxadiazol-2-yl)-7a,8,13,14-tetrahydro-7H- |
| | [1,2,4]triazolo[4′,3′:1,6]pyrido[3,2-b]benzofuro[4,3-fg][1,4]oxazonine |
| 62 | (S)-4-(3,5-dimethylisoxazol-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 63 | (S)-12-fluoro-4-(1-(2-methoxyethyl)-3,5-dimethyl-1H-pyrazol-4-yl)-7a,8,13,14- |
| | tetrahydro-7H-[1,2,4]triazolo[4′,3′:1,6]pyrido[3,2-b]benzofuro[4,3- |
| | fg][1,4]oxazonine |
| 64 | (S)-12-fluoro-N-methyl-N-(tetrahydro-2H-pyran-4-yl)-7a,8,13,14-tetrahydro- |
| | 7H-[1,2,4]triazolo[4′,3′:1,6]pyrido[3,2-b]benzofuro[4,3-fg][1,4]oxazonine-4- |
| | carboxamide |
| 65 | (S)-12-fluoro-4-(5-methylpyrimidin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 66 | (S)-12-fluoro-4-(6-methylpyridazin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 67 | (S)-4-(4,6-dimethylpyridazin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 68 | (S)-4-(4-(difluoromethyl)-2-methylpyrimidin-5-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 69 | (S)-4-(2-(difluoromethyl)-4-methylpyrimidin-5-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 70 | (S)-4-(4-(difluoromethyl)pyrimidin-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 71 | (S)-4-(1,4-dimethyl-1H-pyrazol-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 72 | (S)-4-(1-ethyl-1H-pyrazol-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 73 | (S)-12-fluoro-4-(1-(2,2,2-trifluoroethyl)-1H-pyrazol-5-yl)-7a,8,13,14-tetrahydro- |
| | 7H-[1,2,4]triazolo[4′,3′:1,6]pyrido[3,2-b]benzofuro[4,3-fg][1,4]oxazonine |
| 74 | (S)-1-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1H-pyrazol-1-yl)-2-methylpropan-2-ol |
| 75 | (S)-12-fluoro-4-(1-isopropyl-1H-pyrazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 76 | (S)-4-(2-(difluoromethoxy)pyrimidin-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 77 | (S)-12-fluoro-4-(2-methoxypyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 78 | (S)-4-(2-(difluoromethoxy)pyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 79 | (S)-12-fluoro-4-(3-methyl-1H-pyrazol-1-yl)-7a,8,13,14-tetrahydro-7H- |
| | [1,2,4]triazolo[4′,3′:1,6]pyrido[3,2-b]benzofuro[4,3-fg][1,4]oxazonine |
| 80 | (S)-12-fluoro-4-(4-methyl-1H-pyrazol-1-yl)-7a,8,13,14-tetrahydro-7H- |
| | [1,2,4]triazolo[4′,3′:1,6]pyrido[3,2-b]benzofuro[4,3-fg][1,4]oxazonine |
| 81 | (S)-1-(3-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)pyridin-2-yl)-2-methylpropan-2-ol |
| 82 | (S)-4-(3-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)pyridin-2-yl)-2-methylbutan-2-ol |
| 83 | (S)-12-fluoro-4-(2-(trifluoromethoxy)pyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 84 | (S)-4-(6-(difluoromethyl)-2-methylpyridin-3-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 85 | (S)-4-(2-(difluoromethyl)-6-methylpyridin-3-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 86 | (S)-4-(4,6-dimethylpyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 87 | (S)-12-fluoro-4-(3-fluoro-2-methylpyridin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 88 | (S)-4-(4-(difluoromethyl)-6-methylpyridin-3-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 89 | (S)-12-fluoro-4-(5-fluoro-2-methylpyridin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 90 | (S)-4-(6-(difluoromethyl)-4-methylpyridin-3-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 91 | (S)-12-fluoro-4-(pyrimidin-5-yl)-7a,8,13,14-tetrahydro-7H- |
| | [1,2,4]triazolo[4′,3′:1,6]pyrido[3,2-b]benzofuro[4,3-fg][1,4]oxazonine |
| 92 | (S)-12-fluoro-4-(3-methylisoxazol-4-yl)-7a,8,13,14-tetrahydro-7H- |
| | [1,2,4]triazolo[4′,3′:1,6]pyrido[3,2-b]benzofuro[4,3-fg][1,4]oxazonine |
| 93 | (S)-12-fluoro-4-(thiazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 94 | (S)-12-fluoro-4-(6-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 95 | (S)-12-fluoro-4-(3-methylpyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 96 | (S)-4-(2-ethylpyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 97 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methylpyridin-2(1H)-one |
| 98 | (S)-12-fluoro-4-(6-methoxypyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 99 | (S)-12-fluoro-4-(1,3,5-trimethyl-1H-pyrazol-4-yl)-7a,8,13,14-tetrahydro-7H- |
| | [1,2,4]triazolo[4′,3′:1,6]pyrido[3,2-b]benzofuro[4,3-fg][1,4]oxazonine |
| 100 | (S)-4-(3-ethyl-1-methyl-1H-pyrazol-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 101 | (S)-4-(5-chloropyridin-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 102 | (S)-4-(4-cyclopropylpyrimidin-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 103 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-N,N-dimethylpyridin-2-amine |
| 104 | (S)-12-fluoro-4-(6-methoxy-4-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 105 | (S)-12-fluoro-4-(2-methoxy-6-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 106 | (S)-12-fluoro-4-(6-methoxy-2-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 107 | (S)-12-fluoro-4-(2-methoxy-4-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 108 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-N,N-dimethylpyrimidin-2-amine |
| 109 | (S)-4-(2-ethoxypyrimidin-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 110 | (S)-12-fluoro-4-(5-fluoro-6-methoxypyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 111 | (S)-12-fluoro-4-(5-fluoro-2-methoxypyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 112 | (S)-12-fluoro-4-(6-(trifluoromethyl)pyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 113 | (S)-12-fluoro-4-(5-(trifluoromethyl)pyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 114 | (S)-12-fluoro-4-(2-(trifluoromethyl)pyrimidin-5-yl)-7a,8,13,14-tetrahydro-7H- |
| | [1,2,4]triazolo[4′,3′:1,6]pyrido[3,2-b]benzofuro[4,3-fg][1,4]oxazonine |
| 115 | (S)-12-fluoro-4-(1-methyl-3-(trifluoromethyl)-1H-pyrazol-5-yl)-7a,8,13,14- |
| | tetrahydro-7H-[1,2,4]triazolo[4′,3′:1,6]pyrido[3,2-b]benzofuro[4,3- |
| | fg][1,4]oxazonine |
| 116 | (S)-12-fluoro-4-(6-morpholinopyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 117 | (S)-12-fluoro-4-(6-(4-methylpiperazin-1-yl)pyridin-3-yl)-7a,8,13,14-tetrahydro- |
| | 7H-[1,2,4]triazolo[4′,3′:1,6]pyrido[3,2-b]benzofuro[4,3-fg][1,4]oxazonine |
| 118 | (S)-12-fluoro-4-(2-(4-methylpiperazin-1-yl)pyrimidin-5-yl)-7a,8,13,14- |
| | tetrahydro-7H-[1,2,4]triazolo[4′,3′:1,6]pyrido[3,2-b]benzofuro[4,3- |
| | fg][1,4]oxazonine |
| 119 | (S)-12-fluoro-4-(2-(trifluoromethyl)pyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 120 | (S)-12-fluoro-4-(5-fluoro-6-methylpyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 121 | (S)-12-fluoro-4-(2-methylpyridin-3-yl)-7a,8,13,14-tetrahydro-7H- |
| | [1,2,4]triazolo[r,5′:1,6]pyrido[3,2-b]benzofuro[4,3-fg][1,4]oxazonine |
| 122 | (S)-12-fluoro-4-(1-(2,2,2-trifluoroethyl)-1H-pyrazol-3-yl)-7a,8,13,14-tetrahydro- |
| | 7H-[1,2,4]triazolo[4′,3′:1,6]pyrido[3,2-b]benzofuro[4,3-fg][1,4]oxazonine |
| 123 | (S)-12-fluoro-4-(6-methylpyridazin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 124 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-amine |
| 125 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-N,1-dimethyl-1H-pyrazol-3-amine |
| 126 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-N,N,1-trimethyl-1H-pyrazol-3-amine |
| 127 | (S)-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-yl)methanamine |
| 128 | (S)-1-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-yl)-N- |
| | methylmethanamine |
| 129 | (S)-1-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-yl)-N,N- |
| | dimethylmethanamine |
| 130 | (S)-4-(3-(difluoromethyl)-1-methyl-1H-pyrazol-5-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 131 | (S)-2-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-yl)ethan-1-ol |
| 132 | (S)-2-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-yl)-N,N- |
| | dimethylethan-1-amine |
| 133 | (S)-4-(1,2-dimethyl-1H-imidazol-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 134 | (S)-4-(1,4-dimethyl-1H-imidazol-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 135 | (S)-4-(1,4-dimethyl-1H-imidazol-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 136 | (S)-4-(1,5-dimethyl-1H-imidazol-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 137 | (S)-12-fluoro-4-(1-methyl-1H-imidazol-2-yl)-7a,8,13,14-tetrahydro-7H- |
| | [1,2,4]triazolo[4′,3′:1,6]pyrido[3,2-b]benzofuro[4,3-fg][1,4]oxazonine |
| 138 | (S)-12-fluoro-4-(1-methyl-1H-imidazol-4-yl)-7a,8,13,14-tetrahydro-7H- |
| | [1,2,4]triazolo[4′,3′:1,6]pyrido[3,2-b]benzofuro[4,3-fg][1,4]oxazonine |
| 139 | (S)-4-(1,5-dimethyl-1H-imidazol-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 140 | (S)-4-(1,2-dimethyl-1H-imidazol-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 141 | (S)-4-(5-(difluoromethyl)-6-methylpyridin-2-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 142 | (S)-12-fluoro-4-(1H-pyrazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 143 | (S)-1-(3-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1H-pyrazol-1-yl)-2-methylpropan-2-ol |
| 144 | (S)-4-(3-(difluoromethyl)-6-methylpyridin-2-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 145 | (S)-4-(3-ethyl-1-methyl-1H-1,2,4-triazol-5-yl)-12-fluoro-7a,8,13,14-tetrahydro- |
| | 7H-[1,2,4]triazolo[4′,3′:1,6]pyrido[3,2-b]benzofuro[4,3-fg][1,4]oxazonine |
| 146 | (S)-4-(3-ethyl-1-methyl-1H-pyrazol-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 147 | (S)-12-fluoro-4-(1,2,4-trimethyl-1H-imidazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 148 | (S)-12-fluoro-4-(1,4,5-trimethyl-1H-imidazol-2-yl)-7a,8,13,14-tetrahydro-7H- |
| | [1,2,4]triazolo[4′,3′:1,6]pyrido[3,2-b]benzofuro[4,3-fg][1,4]oxazonine |
| 149 | (S)-12-fluoro-4-(4-methylpyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 150 | (S)-12-fluoro-4-(5-methylpyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 151 | (S)-4-(3-chloropyridin-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 152 | (S)-4-(5-chloro-2-methylpyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 153 | (S)-4-(5-chloro-6-methylpyridin-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 154 | (S)-12-fluoro-4-(5-fluoro-6-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 155 | (S)-12-fluoro-4-(2-methylpyridin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 156 | (S)-4-(2,5-dimethylpyridin-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 157 | (S)-4-(3-chloro-2-methylpyridin-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 158 | (S)-4-(3-chloro-5-fluoropyridin-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 159 | (S)-12-fluoro-4-(3-methoxypyridin-2-yl)-7a,8,13,14-tetrahydro-7H- |
| | [1,2,4]triazolo[4′,3′:1,6]pyrido[3,2-b]benzofuro[4,3-fg][1,4]oxazonine |
| 160 | (S)-12-fluoro-4-(pyrimidin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 161 | (S)-12-fluoro-4-(6-methylpyrimidin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 162 | (S)-12-fluoro-4-(5-methylpyrimidin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 163 | (S)-4-(5-chloropyrimidin-4-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 164 | (S)-12-fluoro-4-(5-fluoropyrimidin-4-yl)-7a,8,13,14-tetrahydro-7H- |
| | [1,2,4]triazolo[4′,3′:1,6]pyrido[3,2-b]benzofuro[4,3-fg][1,4]oxazonine |
| 165 | (S)-4-bromo-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 166 | (S)-4-(5-chloro-3-methylpyridin-2-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 167 | (S)-4-(3-(difluoromethoxy)-1-methyl-1H-pyrazol-5-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 168 | (S)-12-fluoro-4-(3-fluoro-1-methyl-1H-pyrazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 169 | (S)-2-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-yl)-2- |
| | methylpropanenitrile |
| 170 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazole-4-carbonitrile |
| 171 | (S)-4-(5,6-dihydro-4H-pyrrolo[1,2-b]pyrazol-3-yl)-12-fluoro-7a,8,13,14- |
| | tetrahydro-7H-[1,2,4]triazolo[4′,3′:1,6]pyrido[3,2-b]benzofuro[4,3- |
| | fg][1,4]oxazonine |
| 172 | (S)-5-fluoro-12-(1-methyl-1H-pyrazol-5-yl)-6,7,15,15a-tetrahydro-1H- |
| | benzofuro[4,3-fg]imidazo[r,2′:1,6]pyrido[3,2-b][1,4]oxazonine-10-carbonitrile |
| 174 | (S)-5-fluoro-12-(1-methyl-1H-pyrazol-5-yl)-6,7,15,15a-tetrahydro-1H- |
| | benzofuro[4,3-fg]imidazo[r,2′:1,6]pyrido[3,2-b][1,4]oxazonine-10-carboxylic |
| | acid |
| 175 | (S)-5-fluoro-12-(1-methyl-1H-pyrazol-5-yl)-6,7,15,15a-tetrahydro-1H- |
| | benzofuro[4,3-fg]imidazo[r,2′:1,6]pyrido[3,2-b][1,4]oxazonine-10-carboxamide |
| 176 | (S)-5-fluoro-12-(2-methylpyridin-3-yl)-6,7,15,15a-tetrahydro-1H-benzofuro[4,3- |
| | fg]imidazo[r,2′:1,6]pyrido[3,2-b][1,4]oxazonine-10-carboxamide |
| 177 | (S)-5-fluoro-12-(2-methylpyridin-3-yl)-6,7,15,15a-tetrahydro-1H-benzofuro[4,3- |
| | fg]imidazo[r,2′:1,6]pyrido[3,2-b][1,4]oxazonine-10-carboxylic acid |
| 178 | (S)-4-(2-cyclopropylpyrimidin-5-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 179 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-2-methylpyridin-3-amine |
| 181 | methyl 4-(12-fluoro-6,8,13,14-tetrahydro-[1,2,4]triazolo[4′,3′:1,6]pyrido[3,2- |
| | c]benzo[g][1,5]oxazonin-4-yl)piperidine-1-carboxylate |
| 182 | 4-(2,4-dimethylpyrimidin-5-yl)-12-fluoro-6,8,13,14-tetrahydro- |
| | [1,2,4]triazolo[4′,3′:1,6]pyrido[3,2-c]benzo[g][1,5]oxazonine |
| 183 | 12-fluoro-4-((1-methyl-1H-pyrazol-4-yl)methyl)-6,8,13,14-tetrahydro- |
| | [1,2,4]triazolo[4′,3′:1,6]pyrido[3,2-c]benzo[g][1,5]oxazonine |
| 184 | (S)-4-(4,5-dimethyl-4H-1,2,4-triazol-3-yl)-12-fluoro-7a,8,13,14-tetrahydro-7H- |
| | [1,2,4]triazolo[4′,3′:1,6]pyrido[3,2-b]benzofuro[4,3-fg][1,4]oxazonine |
| 185 | (R)-12-fluoro-4-(2-methylpyridin-3-yl)-7a,8,13,14-tetrahydro-7H- |
| | [1,2,4]triazolo[4′,3′:1,6]pyrido[3,2-b]benzofuro[4,3-fg][1,4]oxazonine |
| 186 | (S)-5-fluoro-12-(2-methylpyridin-3-yl)-6,7,15,15a-tetrahydro-1H-benzofuro[4,3- |
| | fg]imidazo[1′,2′:1,6]pyrido[3,2-b][1,4]oxazonine-10-carbonitrile |
| 187 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-6-methylpyridin-2-ol |
| 188 | (S)-12-fluoro-4-(oxazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 189 | (S)-12-fluoro-4-(4-methyloxazol-5-yl)-7a,8,13,14-tetrahydro-7H- |
| | [1,2,4]triazolo[4′,3′:1,6]pyrido[3,2-b]benzofuro[4,3-fg][1,4]oxazonine |
| 190 | (S)-4-(2-cyclopropyl-4-methylpyrimidin-5-yl)-12-fluoro-7a,8,13,14-tetrahydro- |
| | 7H-[1,2,4]triazolo[4′,3′:1,6]pyrido[3,2-b]benzofuro[4,3-fg][1,4]oxazonine |
| 191 | (S)-3-(5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-6-methylpyridin-2-yl)-N- |
| | methylpropanamide |
| 192 | (S)-3-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-2-methylpyridin-4-ol |
| 193 | (S)-1-(4-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-3,6-dihydropyridin-1(2H)-yl)ethan-1-one |
| 194 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-6-methylpyridin-3-ol |
| 195 | (S)-(3-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)pyridin-2-yl)methanol |
| 196 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-4-ol |
| 197 | (S)-5-(12-fluoro-7a,8,13,14-tetrahydro-7H-[1,2,4]triazolo[4′,3′:1,6]pyrido[3,2- |
| | b]benzofuro[4,3-fg][1,4]oxazonin-4-yl)-1-methyl-1H-pyrazol-3-ol |
| 198 | 1-(4-(12-fluoro-6,8,13,14-tetrahydro-[1,2,4]triazolo[4′,3′:1,6]pyrido[3,2- |
| | c]benzo[g][1,5]oxazonin-4-yl)piperidin-1-yl)ethan-1-one |

In some embodiments, the compound is:

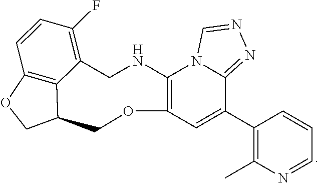 [SMILES: Cc1ncccc1-c1cc2c(n3cnnc13)NCc1c(F)ccc3c1[C@H](CO3)CO2]

In some embodiments, the compound is:

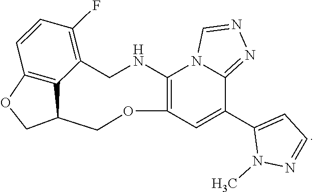 [SMILES: Cn1nccc1-c1cc2c(n3cnnc13)NCc1c(F)ccc3c1[C@H](CO3)CO2]

In some embodiments, the inhibitor is:

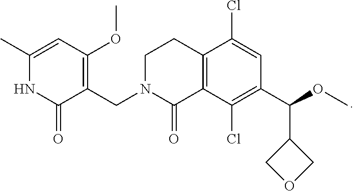

In some embodiments, the inhibitor is:

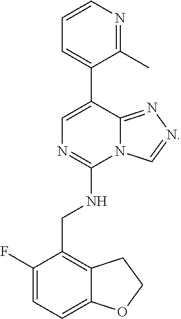 [SMILES: Cc1ncccc1-c1cnc(NCc2c(F)ccc3c2CCO3)n2cnnc12]

Additional inhibitors useful in the methods of the present disclosure include, but are not limited to, tazemetostat, CPI-0209, CPI-1205, EBI-2554, HH-2853, MAK-683, SHR-2554, valemetostat, PF-06821497, ORIC-944, and GSK-2816126.

### Alternative Embodiments

In an alternative embodiment, compounds described herein may also comprise one or more isotopic substitutions. For example, hydrogen may be 2H (D or deuterium) or 3H (T or tritium); carbon may be, for example, 13C or 14C; oxygen may be, for example, 180; nitrogen may be, for example, 15N, and the like. In other embodiments, a particular isotope (e.g., 3H, 13C, 14C, 180, or 15N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### EXAMPLES

Abbreviations: DMSO: dimethyl sulfoxide; EPO: erythropoietin; FPKM: fragments per kilobase of transcript per million mapped reads; HbA: hemoglobin A; HbC: hemoglobin C; HbF: fetal hemoglobin; HbS: hemoglobin S; HPLC: high-performance liquid chromatography; hSCF: human stem cell factor; IMDM: Iscove's Modified Dulbecco's Medium.

### Example 1. Compound 1 Decreases BCL11A mRNA Expression

Human Mobilized Peripheral Blood Primary CD34+ cells from healthy donors were expanded from thaw by seeding 100,000 viable cells/mL in a culture flask containing CD34+ Phase 1 Media comprised of IMDM, 100 ng/ml hSCF, 5 ng/ml IL-3, 3 IU/mL EPO, 250 μg/mL transferrin, 2.5% normal human serum, 1% pen/strep, 10 ng/ml heparin, 10 μg/mL insulin. The cells were supplemented by adding an additional 1X culture volume of CD34+ Phase 1 Media on Day 3 after thaw. At Day 7 post-thaw, cells were seeded at 0.111×10\hat{ }6 cells/ml in 1.5 mLs of Phase 1 media in 12-well plates and compound was applied. On Day 10 post-thaw, cells were differentiated towards the erythroid lineage by complete medium exchange into CD34+ Phase 2 Media comprised of IMDM, 100 ng/ml hSCF, 3 IU/mL EPO, 250 μg/mL transferrin, 2.5% normal human serum, 1% pen/strep, 10 ng/mL heparin, 10 μg/mL insulin and compound treatment was reapplied. On Day 12 post-thaw the plates were centrifuged, and 1 mL of Phase 2 media exchanged with fresh Phase 2 media and compound. The cells were harvested on Day 14 post-thaw for downstream analyses.

RNA was extracted from Day 14 post-thaw primary CD34+ cells that were differentiated and treated for 7 days with DMSO, 11 nM, 33 nM, 100 nM, or 300 nM of Compound 1 using Ambion mirVana RNA extraction kits. Purified RNA samples were submitted for library preparation and deep sequencing at Novogene. Sequenced raw reads of fastq files from all samples were mapped to hg38 genome assemblies using ArrayStudio aligner. Raw read counts and FPKM were calculated for all genes and DESeq2 was applied to calculate differentially expressed genes using general linear model. The statistical cutoff of absolute fold change greater than 4 (fdr<0.05, abs(FC)>4, log2(FPKM+1)>1).

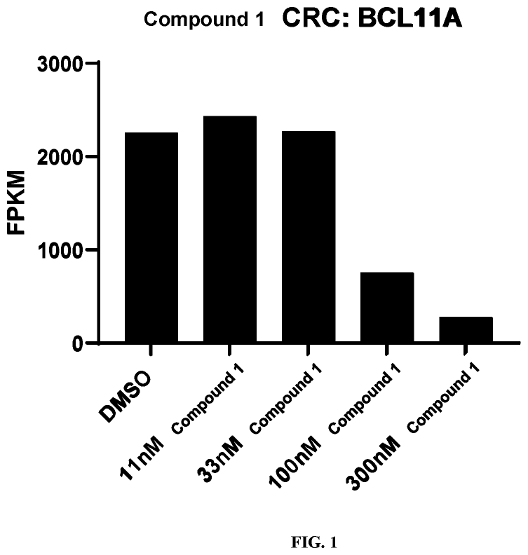

BCL11A mRNA decreased in a concentration dependent manner with Compound 1 challenge, as depicted in FIG. 1. For example, 100 nM Compound 1 significantly decreased BCL11A mRNA levels relative to DMSO and other HbF inducing mechanisms.

### Example 2. EED Inhibitors Decrease BCL11A Expression, Increase HBG1 Expression, and Increase HBG2 Expression in Multiple Healthy CD34+ Donor Cells

Changes in BCL11A expression and HBG1 and HBG2 expression in multiple healthy CD34+ donor cells were measured by RNA sequencing (RNA-Seq). Human Mobilized Peripheral Blood Primary CD34+ cells from 2 healthy donors and 1 sickle cell trait donor were expanded from thaw by seeding 100,000 viable cells/mL in a culture flask containing CD34+ Phase 1 Media comprised of IMDM, 100 ng/ml hSCF, 5 ng/ml IL-3, 3 IU/mL EPO, 250 μg/mL transferrin, 2.5% normal human serum, 1% pen/strep, 10 ng/ml heparin, 10 μg/mL insulin. The cells were supplemented by adding an additional 1X culture volume of CD34+ Phase 1 Media on Day 3 after thaw. At Day 7 post-thaw, cells were seeded at 0.111×10\hat{ }6 cells/ml in 1.5 mLs of Phase 1 media in 12-well plates and compound was applied. On Day 10 post-thaw, cells were differentiated towards the erythroid lineage by complete medium exchange into CD34+ Phase 2 Media comprised of IMDM, 100 ng/ml hSCF, 3 IU/mL EPO, 250 μg/mL transferrin, 2.5% normal human serum, 1% pen/strep, 10 ng/ml heparin, 10 μg/mL insulin and compound treatment was reapplied. On Day 12 post-thaw the plates were centrifuged, and 1 mL of Phase 2 media exchanged with fresh Phase 2 media and compound. The cells were harvested on Day 14 post-thaw for downstream analyses.

RNA was extracted from Day 14 post-thaw primary CD34+ cells that were differentiated and treated for 7 days with DMSO, 100 nM of Compound 1, or 100 nM of Compound 2 using Ambion mirVana RNA extraction kits. Purified RNA samples were submitted for library preparation and deep sequencing at Novogene. Sequenced raw reads of fastq files from all samples were mapped to hg38 genome assemblies using ArrayStudio aligner. Raw read counts and FPKM were calculated for all genes and DESeq2 was applied to calculate differentially expressed genes using general linear model. The statistical cutoff of absolute fold change greater than 4 (fdr<0.05, abs(FC)>4, log2(FPKM+1)>1).

Hemoglobin protein tetramers were analyzed using HPLC analysis following a protocol derived from Gravett et al. Science 2018. Briefly, approximately 1 million cells were lysed in 100 uL of water with 10 uL of lysate used per injection. Hemolysates were cleared by centrifugation and analyzed for identity and levels of hemoglobin variants (HbF and HbA) by cation-exchange HPLC with a weak cation-exchange column (Poly CAT A: 35 mm×4.6 mm, Poly LC, Inc., Columbia, MD). Hemoglobin isotype peaks were eluted with a linear gradient of phase B from 0% to 80% at A410 nm (Mobile Phase A: 20 mM Bis-Tris, 2 mM KCN, pH 6.95; Phase B:20 mM Bis-Tris, 2 mM KCN, 0.2 M sodium chloride, pH 6.55). Species were monitored with UV absorbance at 410 nm. Retention times for hemoglobin species was optimized using the hemoglobin FASC control (PerkinElmer) as isotype controls for HbF, HbA, HbS and HbC. The abundance of HbF, HbS, and HbA was quantified by calculating the area under the curve for each species in the samples. % HbF was calculated as HbF/(HbF+HbS+HbA)*100.

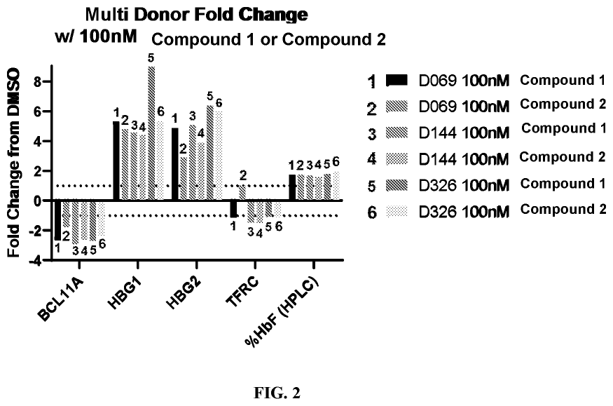

100 nM of Compound 1 and 100 nM of Compound 2 decreased BCL11A and increase HBG1 and HBG2 expression in all donors studied (i.e., D069, D144, and D326 donors), as shown in FIG. 2. It was observed that expression of the housekeeping gene TFRC did not result in substantial changes in expression upon exposure to Compound 1 or Compound 2. However, an approximately 2-fold increase in percentage HbF by HPLC at 100 nM Compound 1 or 100 nM Compound 2.

### Example 3. Dependence of Compound 1 Concentration on BCL11A Expression, HBG1 Expression, HBG2 Expression, and Percentage HbF

A single donor (D069) was treated with Compound 1 to generate 4-point concentration response curves of fold changes in BCL11A expression, HBG1 expression, and HBG2 expression, and percentage HbF levels. Human Mobilized Peripheral Blood Primary CD34+ cells from 1 healthy donor were expanded from thaw by seeding 100,000 viable cells/mL in a culture flask containing CD34+ Phase 1 Media comprised of IMDM, 100 ng/ml hSCF, 5 ng/mL IL-3, 3 IU/mL EPO, 250 μg/mL transferrin, 2.5% normal human serum, 1% pen/strep, 10 ng/ml heparin, 10 μg/mL insulin. The cells were supplemented by adding an additional 1X culture volume of CD34+ Phase 1 Media on Day 3 after thaw. At Day 7 post-thaw, cells were seeded at 0.111×10\hat{ }6 cells/ml in 1.5 mLs of Phase 1 media in 12-well plates and compound was applied. On Day 10 post-thaw, cells were differentiated towards the erythroid lineage by complete medium exchange into CD34+ Phase 2 Media comprised of IMDM, 100 ng/ml hSCF, 3 IU/mL EPO, 250 μg/mL transferrin, 2.5% normal human serum, 1% pen/strep, 10 ng/ml heparin, 10 μg/mL insulin and compound treatment was reapplied. On Day 12 post-thaw the plates were centrifuged, and 1 mL of Phase 2 media exchanged with fresh Phase 2 media and compound. The cells were harvested on Day 14 post-thaw for downstream analyses.

RNA was extracted from Day 14 post-thaw primary CD34+ cells that were differentiated and treated for 7 days with DMSO, 11 nM, 33 nM, 100 nM, or 300 nM of Compound 1 using Ambion mirVana RNA extraction kits. Purified RNA samples were submitted for library preparation and deep sequencing at Novogene. Sequenced raw reads of fastq files from all samples were mapped to hg38 genome assemblies using ArrayStudio aligner. Raw read counts and FPKM were calculated for all genes and DESeq2 was applied to calculate differentially expressed genes using general linear model. The statistical cutoff of absolute fold change greater than 4 (fdr<0.05, abs(FC)>4, log2(FPKM+1)>1).

Hemoglobin protein tetramers were analyzed using HPLC analysis following a protocol derived from Gravett et al. Science 2018. Briefly, approximately 1 million cells were lysed in 100 uL of water with 10 uL of lysate used per injection. Hemolysates were cleared by centrifugation and analyzed for identity and levels of hemoglobin variants (HbF and HbA) by cation-exchange HPLC with a weak cation-exchange column (Poly CAT A: 35 mm×4.6 mm, Poly LC, Inc., Columbia, MD). Hemoglobin isotype peaks were eluted with a linear gradient of phase B from 0% to 80% at A410 nm (Mobile Phase A: 20 mM Bis-Tris, 2 mM KCN, pH 6.95; Phase B:20 mM Bis-Tris, 2 mM KCN, 0.2 M sodium chloride, pH 6.55). Species were monitored with UV absorbance at 410 nm. Retention times for hemoglobin species was optimized using the hemoglobin FASC control (PerkinElmer) as isotype controls for HbF, HbA, HbS and HbC. The abundance of HbF, HbS, and HbA was quantified by calculating the area under the curve for each species in the samples. % HbF was calculated as HbF/(HbF+HbS+HbA)*100.

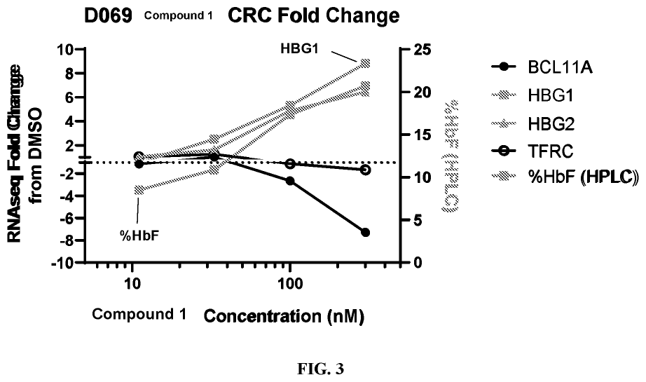

A decrease in BCL11A expression, an increase in HBG1 expression, an increase in HBG2 expression, and an increase in HbF protein levels were observed in a concentration-dependent fashion and at similar concentrations, as shown in FIG. 3.

### Example 4. PRC2 Inhibition Reduces Expression of BCL11A Resulting in Induction of HBG1 Expression, Induction of HBG2 Expression, and Percentage HbF

The effect of PRC2 inhibition on BCL11A expression, HBGI expression, HBG2 expression, and percentage HbF in two different CD34+ donors was studied. Human Mobilized Peripheral Blood Primary CD34+ cells from 2 healthy donors were expanded from thaw by seeding 100,000 viable cells/mL in a culture flask containing CD34+ Phase 1 Media comprised of IMDM, 100 ng/mL hSCF, 5 ng/ml IL-3, 3 IU/mL EPO, 250 μg/mL transferrin, 2.5% normal human serum, 1% pen/strep, 10 ng/ml heparin, 10 μg/mL insulin. The cells were supplemented by adding an additional 1× culture volume of CD34+ Phase 1 Media on Day 3 after thaw. At Day 7 post-thaw, cells were seeded at 0.111×10\hat{ }6 cells/ml in 1.5 mLs of Phase 1 media in 12-well plates and compound was applied. On Day 10 post-thaw, cells were differentiated towards the erythroid lineage by complete medium exchange into CD34+ Phase 2 Media comprised of IMDM, 100 ng/mL hSCF, 3 IU/mL EPO, 250 μg/mL transferrin, 2.5% normal human serum, 1% pen/strep, 10 ng/ml heparin, 10 μg/mL insulin and compound treatment was reapplied. On Day 12 post-thaw the plates were centrifuged, and 1 mL of Phase 2 media exchanged with fresh Phase 2 media and compound. The cells were harvested on Day 14 post-thaw for downstream analyses.

RNA was extracted from Day 14 post-thaw primary CD34+ cells that were differentiated and treated for 7 days with DMSO, Compound 1 (11 nM, 33 nM, 100 nM, 300 nM), Compound 2 (11 nM, 33 nM, 100 nM, 300 nM), Compund 3 (37 nM, 111 nM, 333 nM, 1 uM), Compound 4 (37 nM, 111 nM, 333 nM, 1 uM) using Ambion mirVana RNA extraction kits. Purified RNA samples were submitted for library preparation and deep sequencing at Novogene. Sequenced raw reads of fastq files from all samples were mapped to hg38 genome assemblies using ArrayStudio aligner. Raw read counts and FPKM were calculated for all genes and DESeq2 was applied to calculate differentially expressed genes using general linear model. The statistical cutoff of absolute fold change greater than 4 (fdr<0.05, abs(FC)>4, log2(FPKM+1)>1).

Hemoglobin protein tetramers were analyzed using HPLC analysis following a protocol derived from Gravett et al. Science 2018. Briefly, approximately 1 million cells were lysed in 100 uL of water with 10 uL of lysate used per injection. Hemolysates were cleared by centrifugation and analyzed for identity and levels of hemoglobin variants (HbF and HbA) by cation-exchange HPLC with a weak cation-exchange column (Poly CAT A: 35 mm×4.6 mm, Poly LC, Inc., Columbia, MD). Hemoglobin isotype peaks were eluted with a linear gradient of phase B from 0% to 80% at A410 nm (Mobile Phase A: 20 mM Bis-Tris, 2 mM KCN, pH 6.95; Phase B:20 mM Bis-Tris, 2 mM KCN, 0.2 M sodium chloride, pH 6.55). Species were monitored with UV absorbance at 410 nm. Retention times for hemoglobin species was optimized using the hemoglobin FASC control (PerkinElmer) as isotype controls for HbF, HbA, HbS and HbC. The abundance of HbF, HbS, and HbA was quantified by calculating the area under the curve for each species in the samples. % HbF was calculated as HbF/(HbF+HbS+HbA)*100.

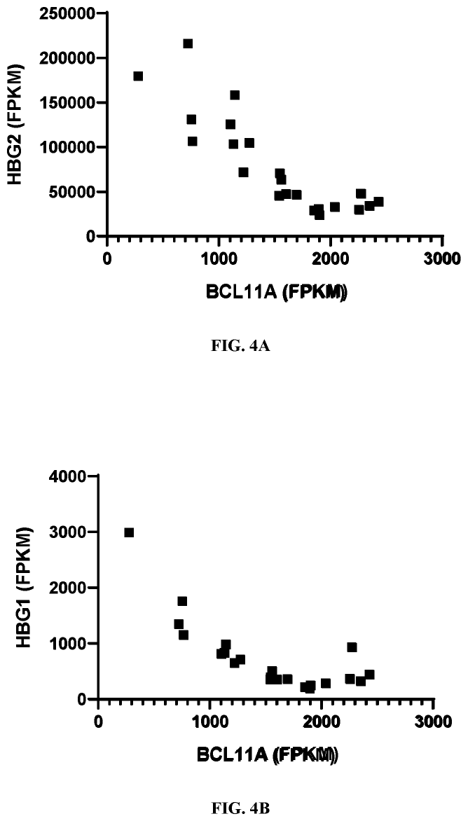

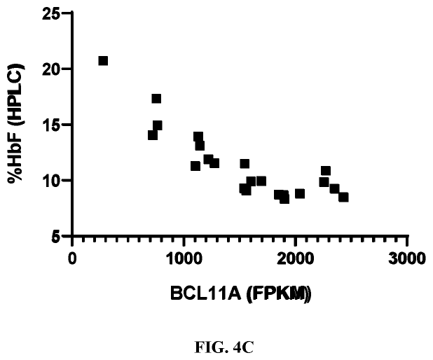

From the procedure, aggregate data from two different CD34+ donors (D069 and D301) and multiple compounds that inhibit PRC2, particularly Compound 1, Compound 2, Compound 3, and Compound 4 were collected, as shown in FIG. 4A, FIG. 4B, and FIG. 4C.

The data show that HBG1 mRNA expression level (FIG. 4A), HBG2 mRNA expression level (FIG. 4B), and percentage HbF protein (FIG. 4C) correlate to a decrease in BCL11A mRNA levels regardless of donor, concentration, or PRC2 inhibitor selected.

### Example 5. Effect of Various PRC2 Inhibitors on BCL11A, HBG1, and HBG2 Expression

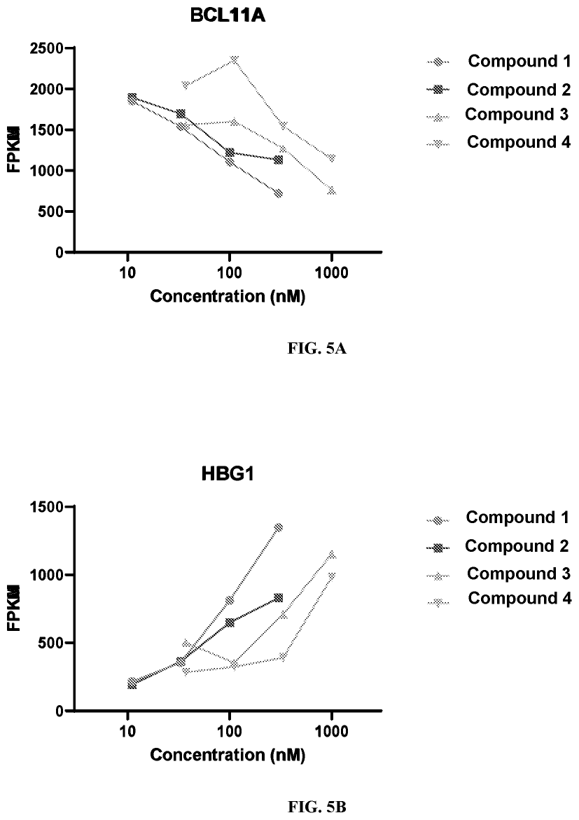

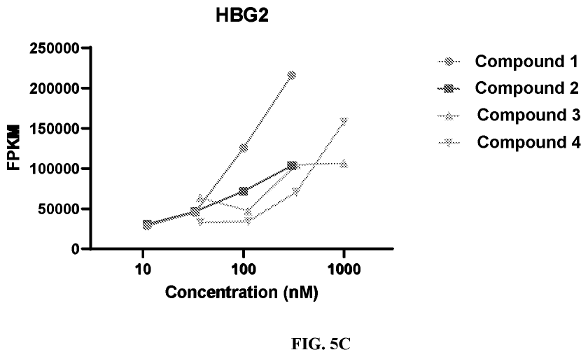

4-point concentration response curves of the PRC2 inhibitors Compound 1, Compound 2, Compound 3, and Compound 4 on BCL11A expression (FIG. 5A), HBG1 expression (FIG. 5B), and HBG2 expression (FIG. 5C), were generated. The data show that all PRC2 inhibitors profiled show a reduction in BCL11A expression and a concomitant induction of HBG1 and HBG2 expression.

### Example 6. Studies of Effects of Compound 1 and Gene Knockdown BCL11A mRNA Levels

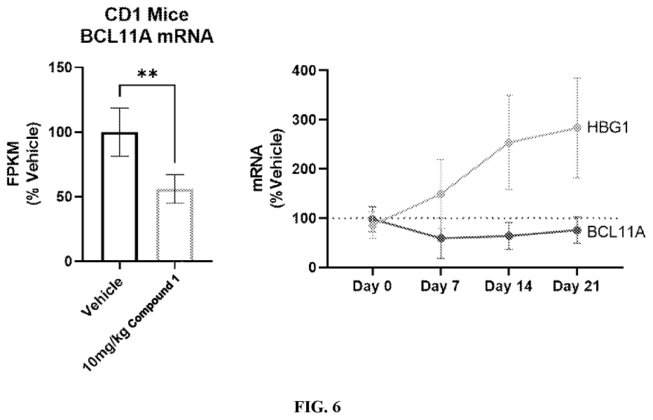

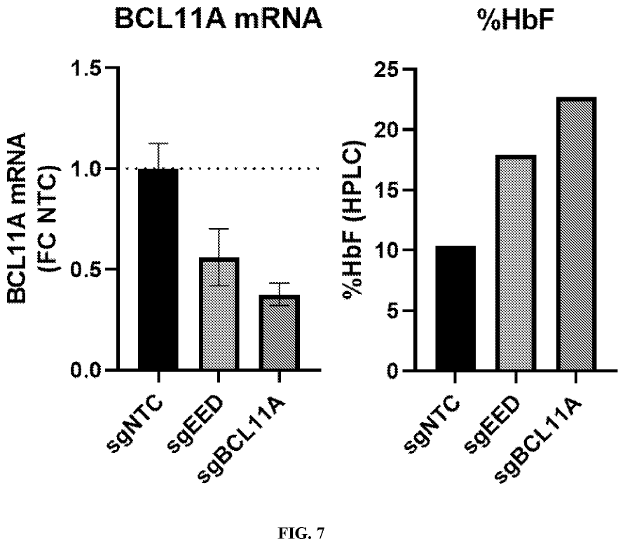

Whole blood of wildtype CD-1 mice were treated for 5 days with 10 mg/kg Compound 1, and which was maintained over time in the Townes sickle cell disease (SCD) mouse model (FIG. 6). Furthermore, CRISPR-Cas9 in CD34+ cells was used to understand if loss-of-function of EED or BCL11A recapitulated the pharmacologic impact on fetal hemoglobin expression with Compound 1. Knockdown in vitro of both BCL11A and EED genes showed a significant reduction in BCL11A mRNA levels with a corresponding induction of HbF protein (FIG. 7).

Whole Blood Real-Time Quantitative PCR (qRT-PCR)

Whole blood was collected and preserved in DNA/RNA Shield Solution. RNA was extracted with MagMAX mirVana Total RNA Isolation Kit with KingFisher™ Flex Purification System. 10 ng of RNA was used for one-step TaqMan RNA-to-Ct reaction using Taqman reverse transcriptase Multiplex Master Mix (ThermoFisher, #4484262) following the manufacturer's recommendations. Amplification was detected in a Quantstudio 7 Flex instrument from ThermoFisher. Transcript specific Taqman probes purchased from ThermoFisher are shown in Table 1. The relative expression levels from each gene target were calculated and normalized to geometric mean of three reference genes (Gapdh, Oazl and Tfrc) using 2^{-4Ct} method.

### Example 7. Comparative Studies of Compound 1 and Hydroxyurea (HU) in an SCD Mouse Model

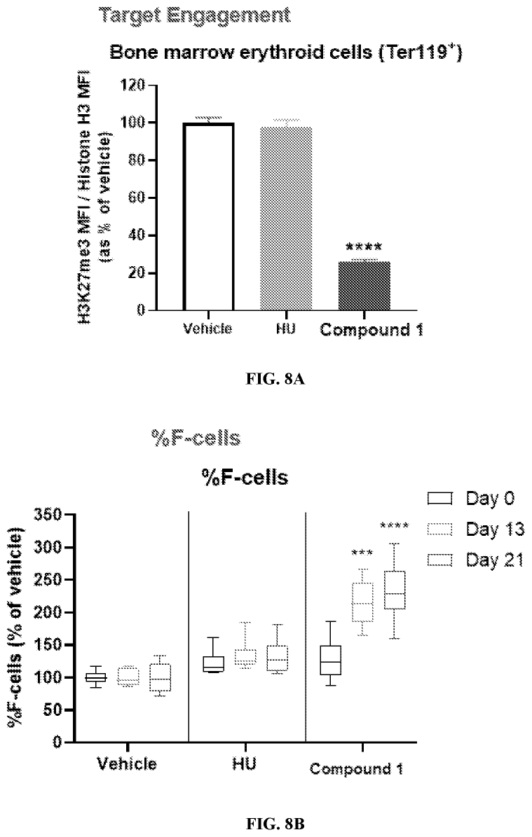

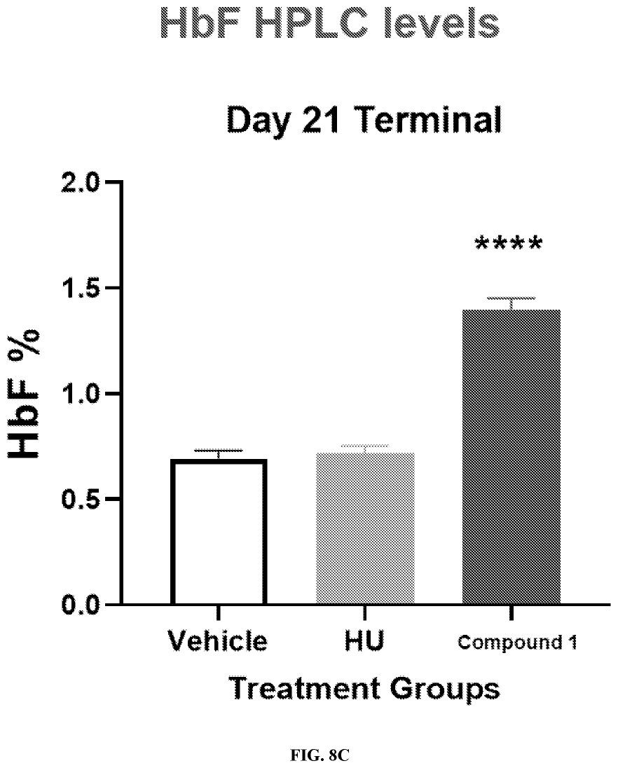

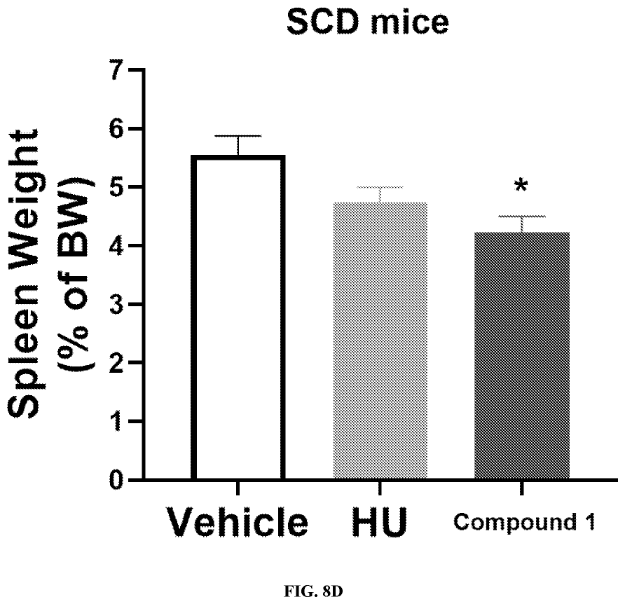

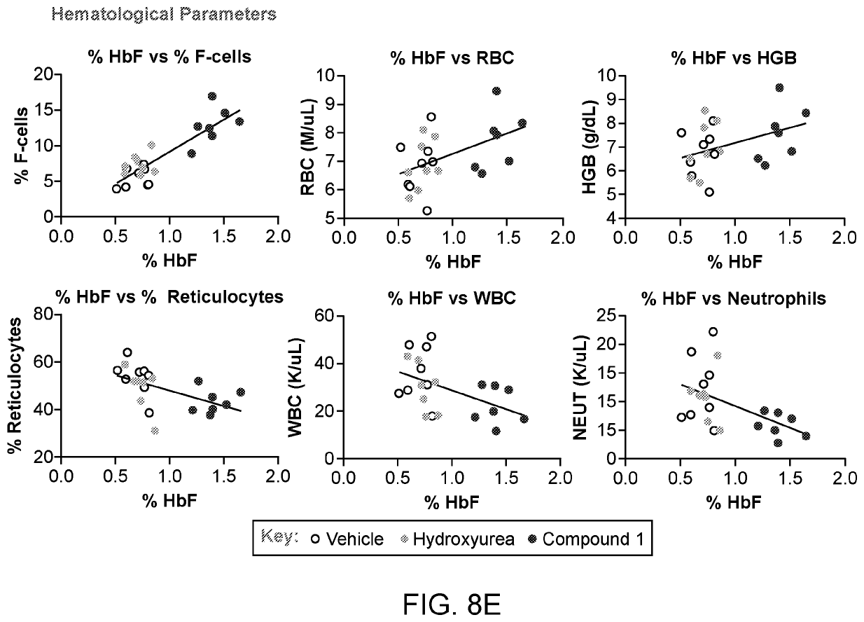

The Townes SCD mouse model was used to study the in vivo pharmacologic activity of Compound 1 in a model with the relevant human globin genes (HBG1 and HBB_{E6V}) integrated into the mouse beta globin locus. Hydroxyurea was used as a benchmark for this 21-day study. The Compound 1 treatment was well-tolerated in the SCD mice and target engagement analysis found that levels of H3K27me3 were significantly reduced in Ter119+ bone marrow cells with Compound 1 treatment but not hydroxyurea treatment after 28 days of treatment as expected (FIG. 8A). Compound 1 treatment led to increases in % F-cells when measured at Day 13 and Day 21, while hydroxyurea did not significantly impact F-cells (FIG. 8B). Despite the Townes SCD mouse having low basal HbF expression, % HbF of total hemoglobin was quantified by HPLC and showed significant induction of HbF with Compound 1 treatment consistent with the 2-3 fold induction observed preclinically, while no change in HbF was observed with hydroxyurea (FIG. 8C). The Compound 1 treated Townes mice showed a statistically significant reduction in spleen weight as a percent of total body weight by Day 21, further demonstrating the benefit of increased HbF on SCD-related symptomatology (FIG. 8D). People living with sickle cell present with impaired erythropoiesis leading to elevated reticulocytes, decreased red blood cell count, and total hemoglobin, and additionally display markers of inflammation such as elevated neutrophils and white blood cells. Similarly, the Townes mouse model recapitulates these hematological aspects of SCD. The Compound 1 induced increase in HbF, although expressed at low levels relative to total hemoglobin, translated to positive improvements in hematological parameters in the Townes mice such as RBC count, total Hb, % reticulocytes, WBCs, and neutrophils (FIG. 8E).

### Equivalents

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method of downregulating BCL11A in a cell comprising: contacting the cell sample with an EED, EHZ2, and/or PRC2 inhibitor in amount sufficient to decrease expression of BCL11A.

2. The method of claim 1, wherein the cell is an erythroid cell differentiated from a CD34+ cell.

3. The method of claim 2, wherein upon contacting the cell, HBG1 and/or HBG2 expression increases.

4. The method of any one of claims 1-3, wherein upon contacting the cell with the protein inhibitor, the cell does or does not express fetal hemoglobin.

5. A method of identifying a patient having a BCL11A mediated disorder who may benefit from treatment comprising one or more inhibitors of EED, EHZ2, and/or PRC2, comprising determining an expression level of BCL11A in a sample obtained from the patient, wherein an increased expression level of BCL11A in the sample as compared to a reference expression level identifies the patient as one who may benefit from the EED, EHZ2, and/or PRC2 inhibitor treatment.

6. A method of treating a patient having a BCL11A mediated disorder, comprising administering to the patient a therapeutically effective amount of a EED, EHZ2, and/or PRC2 inhibitor, wherein the expression level of BCL11A in a sample obtained from the patient has been determined to be decreased after the administration as compared to a reference expression level.

7. The method of any one of claims 1-6, wherein the EED, EHZ2, and/or PRC2 inhibitor is selected from the group consisting of an antibody against EED, EHZ2, and/or PRC2 or an antigen-binding fragment thereof, a small molecule, and a nucleic acid.

8. The method of claim 7, wherein the nucleic acid is a EED, EHZ2, and/or PRC2-specific RNA interference agent, a vector encoding a RNA interference agent, or an aptamer that binds EED, EZH2, and/or PRC2.

9. The method of any one of claims 5-8, wherein the BCL11A mediated disorder is selected from the group consisting of triple negative breast cancer, non-small cell lung cancer, glioblastoma, neuroblastoma, prostate cancer, type 2 diabetes, laryngeal squamous cell carcinoma, and Williams syndrome.

10. A method of treatment of a hemoglobinopathy in a subject comprising administering an effective amount of a composition comprising an inhibitor of EED, EHZ2, and/or PRC2, wherein the inhibitor of PRC2 downregulates the expression of BCL11A and whereby fetal hemoglobin expression is increased in the subject relative to prior to the administration.

11. The method of claim 10, wherein the hemoglobinopathy is selected from the group consisting of sickle cell disease (SCD), α-thalassemia, and β-thalassemia.

12. The method of claim 11, wherein the β-thalassemia is selected from the group consisting of sickle β-thalassemia, hemoglobin C β-thalassemia, and hemoglobin E β-thalassemia.

13. The method of claim 12, wherein sickle β-thalassemia is selected from sickle β0 thalassemia and sickle β+thalassemia.

14. The method of any one of claims 1-13, wherein the expression level of BCL11A is decreased by at least 25% relative to a reference level.

15. The method of any one of claims 1-14, wherein the expression level is a protein expression level or a mRNA expression level.

16. The method of claim 15, wherein the mRNA expression level is determined by qPCR or RNA-Seq.

17. The method of claim 15, wherein the protein expression level is determined using a method selected from the group consisting of high-performance liquid chromatography (HPLC), immunohistochemistry (IHC), immunofluorescence, mass spectrometry, flow cytometry, and Western blot.

18. The method of any one of claims 1-17, wherein the EED, EHZ2, and/or PRC2 inhibitor is selected from the group consisting of: 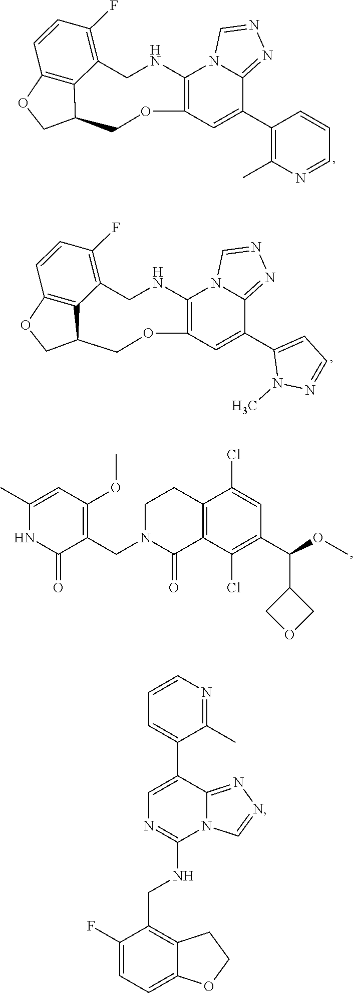
tazemetostat, CPI-0209, CPI-1205, EBI-2554, HH-2853, MAK-683, SHR-2554, valemetostat, PF-06821497, ORIC-944, and GSK-2816126.

19. A genetically modified cell comprising an insertion and/or deletion in a gene loci that encodes a protein selected from the group consisting of EED, EHZ2, and PRC2, wherein the insertion and/or deletion is capable of downregulating expression of BCL11A in the cell.

20. The genetically modified cell of claim 19, wherein the expression level of BCL11A is decreased by at least 25% relative to a reference level.

21. The genetically modified cell of claim 19 or 20, wherein the expression level is a protein expression level or a mRNA expression level.

22. The genetically modified cell of claim 21, wherein the mRNA expression level is determined by qPCR or RNA-Seq.

23. The genetically modified cell of claim 21, wherein the protein expression level is determined using a method selected from the group consisting of high-performance liquid chromatography (HPLC), immunohistochemistry (IHC), immunofluorescence, mass spectrometry, flow cytometry, and Western blot.
